(19) United States
(12) Patent Application Publication
(10) Pub. No.: US 2021/0343422 A1
(43) Pub. Date: Nov. 4, 2021

(54) METHODS FOR DYNAMIC MODELING AND CLOSED-LOOP CONTROL OF INFLAMMATION

(71) Applicant: Georgia Tech Research Corporation, Atlanta, GA (US)

(72) Inventors: Levi Wood, Atlanta, GA (US); James E. Forsmo, Atlanta, GA (US); Laura D. Weinstock, Atlanta, GA (US); Alexis F. Wilkinson, Atlanta, GA (US)

(21) Appl. No.: 17/284,216

(22) PCT Filed: Oct. 11, 2019
(86) PCT No.: PCT/US2019/055821

§ 371 Date: Apr. 9, 2021

Related U.S. Application Data
(60) Provisional application No. 62/744,760, filed on Oct. 12, 2018.

(51) Int. Cl. G16H 50/50; G01N 33/50

(52) U.S. Cl. CPC G16H 50/50; G01N 33/5047

(57) ABSTRACT

The disclosure is directed to technologies for restoring proper regulation of the immune response and novel methods and systems for exogenously controlling immune cells in order to dynamically and predictively drive the immune response through pro-inflammatory activity to anti-inflammatory activity, mimicking the immune system's natural progression through these states. Embodiments of the present disclosure relate generally to methods and systems for dynamic predictive modeling and control of inflammation and the immune response, and more specifically to methods and systems for predictive modeling and control of the inflammatory state of immune cells via temporally regulated immune-modulating stimuli.

### CROSS-REFERENCE TO RELATED APPLICATIONS

This application claims priority to U.S. Provisional Application No. 62/744,760, filed on 12 Oct. 2018, the disclosure of which is herein incorporated by reference in its entirety.

### GOVERNMENT SPONSORSHIP

This invention was made with government support under Grant No. T32-GM008433 awarded by the National Institutes of Health. The government has certain rights in the invention.

### BACKGROUND OF THE DISCLOSURE

### 1. Field of the Disclosure

Embodiments of the present disclosure relates generally to methods and systems for dynamic predictive modeling and control of inflammation and the immune response, and more specifically to methods and systems for predictive modeling and control of the inflammatory state of immune cells via temporally regulated immune-modulating stimuli.

### 2. Background

Healthy immune response during infection or injury is a dynamic process consisting of initial, acute pro-inflammatory activity followed by an anti-inflammatory/resolving state, both requiring macrophages as major mediators. This temporally regulated response promotes pathogen and debris clearance followed by tissue regeneration and, ultimately, recovery of homeostasis (FIGS. 1A-1B). Dysregulation of the immune response can occur in many ways and can last for a short time or become chronic. Broad ablation of immune response, e.g., via corticosteroids, can equally limit successful regeneration and recovery of tissue homeostasis. There is thus an urgent need to gain a nuanced understanding of tissue immune response and how regulation can be regained when endogenous regulation is lost.

Although the need for regulation of tissue immune response is well-recognized, identification of new strategies to intervene in tissue inflammatory response remain a major challenge. Dynamic immune response by macrophages and other immune cells is integral to both the early (<1 hr) and continued (>1 month) response to infections and injury. Without appropriate regulation of their activity, macrophages and other immune cells can drive the initiation and progression of many diseases. In particular, loss of regulation can lead to insufficient pro-inflammatory activity leading to incomplete clearance of pathogens and/or tissue debris, impaired pro-regenerative response, chronic inflammation, and infection. Recent efforts to regulate dysfunctional macrophages have focused on cell-based therapies, such as delivery of mesenchymal stem cells (MSCs) or macrophages conditioned ex vivo toward anti-inflammatory and pro-regenerative “M2” phenotypes. The underlying principal behind immunomodulatory cell therapies is that these cells will act as natural “controllers” of immune response through beneficial immunomodulatory signaling in the local environment. However, these strategies are subject to a number of limitations. For example, MSCs are subject to variable efficacy between donors and batches. Other approaches seek to deliver ex vivo modified macrophages, but both mouse and human trials have had variable success and still face many challenges. A new approach that actively regulates resident tissue macrophages could escape many challenges faced by current cell-based therapies.

Exogenous control of macrophage and immune cell activity could provide a new method to modulate the immune response that would steer the system through a desired trajectory of activity akin to the autopilot in an airplane. Macrophages are an attractive target for regulating immune response because i) they are involved in diverse immune functions essential for tissue protection and repair and ii) they are highly plastic with the ability to dynamically re-polarize for different functions based on external cues. Since macrophage polarization is dynamic, a quantitative temporal model could enable design of exogenous input sequences capable of normalizing response (FIGS. 1A-1C). The pathways governing macrophage polarization in response to stimuli have been comprehensively modeled, including receptor binding kinetics, downstream kinase signaling, and gene transcription. While mechanistically appealing, these models possess dozens of equations and hundreds of parameters, making it intractable to identify reliably predictive input-output relationships between exogenous stimulation and polarization in terms of these precise mechanistic models. Moreover, it has recently been argued that identification of viable strategies to intervene in immune activity will require rigorous integration of experimental data with computational modeling. There is thus a need for an empirical input/output model that relates macrophage response to exogenous inputs in order to predict and control activation levels over time.

What is needed, therefore, are methods and systems for exogenously controlling immune cells in order to dynamically and predictively drive the immune response through pro-inflammatory activity to anti-inflammatory activity, mimicking the immune system's natural progression through these states during health and restoring proper regulation. These methods and systems should enable predictive monitoring and control of immune cell polarization, leading to predictive control and regulation of inflammation and the immune response. The methods and systems should also enable predictive modeling and control of the inflammatory state of immune cells via temporally regulated immune-modulating stimuli. It is to such a method and system that embodiments of the present disclosure are directed.

### BRIEF SUMMARY OF THE DISCLOSURE

As specified in the Background Section, there is a great need in the art to identify technologies for restoring proper regulation of the immune response and use this understanding to develop novel methods and systems for exogenously controlling immune cells in order to dynamically and predictively drive the immune response through pro-inflammatory activity to anti-inflammatory activity, mimicking the immune system's natural progression through these states during health. The present disclosure satisfies this and other needs. Embodiments of the present disclosure relate generally to methods and systems for dynamic predictive modeling and control of inflammation and the immune response, and more specifically to methods and systems for predictive modeling and control of the inflammatory state of immune cells via temporally regulated immune-modulating stimuli.

In one aspect, the disclosure provides a method for dynamic real-time modeling and/or control of an inflammatory response in an immune cell, comprising: providing a fluid chamber comprising at least one inlet, at least one outlet, and the immune cell; delivering a first stimulus through the inlet via a controller, the controller in fluid communication with the fluid chamber, wherein the stimulus elicits a change in an inflammatory state of the immune cell; and detecting the change in the inflammatory state of the immune cell via a detector, the detector in fluid communication with the fluid chamber, wherein the controller is configured to deliver a second stimulus based on the change in the inflammatory state of the immune cell in order to model and/or control the inflammatory response of the immune cell, wherein the detector is configured to generate input and/or output data indicative of the change in the inflammatory state of the immune cell, and wherein the change in the inflammatory state of the immune cell to each of the first stimulus and second stimulus is predicted by the steps of: fitting a black box engineering model to the input and/or output data obtained by stimulating cells within the chamber; and selecting a best fitting black box engineering model based on the input and/or output data and applying the best fitting black box engineering model to future input and/or output data.

In another aspect, the disclosure provides a method of treating a disease or condition in a subject in need thereof caused by an aberrant inflammatory response comprising: monitoring and/or controlling in real time the aberrant inflammatory response in an immune cell, comprising: providing a fluid chamber comprising at least one inlet, at least one outlet, and the immune cell; delivering a first stimulus through the inlet via a controller, the controller in fluid communication with the fluid chamber, wherein the stimulus elicits a change in an inflammatory state of the immune cell; and detecting the change in the inflammatory state of the immune cell via a detector, the detector in fluid communication with the fluid chamber, wherein the controller is configured to deliver a second stimulus based on the change in the inflammatory state of the immune cell in order to model and/or control the inflammatory response of the immune cell, wherein the detector is configured to generate input and/or output data indicative of the change in the inflammatory state of the immune cell, and wherein the change in the inflammatory state of the immune cell to each of the first stimulus and second stimulus is predicted by the steps of: fitting a black box engineering model to the input and/or output data obtained by stimulating cells within the chamber; and selecting a best fitting black box engineering model based on the input and/or output data and applying the best fitting black box engineering model to future input and/or output data, and wherein the first and/or second stimulus is administered to the subject in order to control the aberrant inflammatory response thereby treating the disease or condition.

In another aspect, the disclosure provides a method of treating a disease or condition in a subject in need thereof caused by an aberrant inflammatory response comprising: administering a first stimulus to the subject, wherein the stimulus elicits a change in an inflammatory state of the subject's immune cells; obtaining a biological sample from the subject; detecting the change in the inflammatory state via a detector; delivering a second stimulus based on the change in the inflammatory state of the immune cell in order to model and/or control the inflammatory response of the immune cells, wherein the detector is configured to generate input and/or output data indicative of the change in the inflammatory state of the immune cells, wherein the change in the inflammatory state of the immune cells to each of the first stimulus and second stimulus is predicted by the steps of: fitting a black box engineering model to the input and/or output data obtained by stimulating the subject's immune cells; and selecting the best fitting black box engineering model based on the input and/or output data and applying the best fitting black box engineering model to future input and/or output data, and wherein the first and/or second stimulus is administered to the subject in order to control the aberrant inflammatory response thereby treating the disease or condition.

In another aspect, the disclosure provides a system for dynamic real-time modeling and/or control of an inflammatory response in an immune cell, comprising: a fluid chamber comprising at least one inlet, at least one outlet, and the immune cell; a controller in fluid communication with the fluid chamber configured to deliver a first stimulus through the inlet, wherein the stimulus elicits a change in the inflammatory state of the immune cell; and a detector in fluid communication with the fluid chamber configured to detect the change in the inflammatory state of the immune cell, wherein the controller is further configured to deliver a second stimulus based on the change in the inflammatory state of the immune cell in order to model and/or control the inflammatory response of the immune cell, wherein the detector is configured to generate input and/or output data indicative of the change in the inflammatory state of the immune cell, and wherein the change in the inflammatory state of the immune cell to each of the first stimulus and second stimulus is predicted by the steps of: fitting a black box engineering model to the input and/or output data obtained by stimulating cells within the chamber; and selecting a best fitting black box engineering model based on the input/output data and applying the best fitting black box engineering model to future input and/or output data.

In another aspect, the disclosure provides a system for treating a disease or condition in a subject in need thereof caused by an aberrant inflammatory response comprising: monitoring and/or controlling in real time the aberrant inflammatory response in an immune cell, comprising: providing a fluid chamber comprising at least one inlet, at least one outlet, and the immune cell; delivering a first stimulus through the inlet via a controller, the controller in fluid communication with the fluid chamber, wherein the stimulus elicits a change in the inflammatory state of the immune cell; and detecting the change in the inflammatory state of the immune cell via a detector, the detector in fluid communication with the fluid chamber, wherein the controller is configured to deliver a second stimulus based on the change in the inflammatory state of the immune cell in order to model and/or control the inflammatory response of the immune cell, wherein the detector is configured to generate input and/or output data indicative of the change in the inflammatory state of the immune cell, wherein the change in the inflammatory state of the immune cell to each of the first stimulus and second stimulus is predicted by the steps of: fitting a black box engineering model to the input and/or output data obtained by stimulating cells within the chamber; and selecting a best fitting black box engineering model based on the input and/or output data and applying the best fitting black box engineering model to future input and/or output data, and wherein the first and/or second stimulus is administered to the subject in order to control the aberrant inflammatory response thereby treating the disease or condition.

These and other objects, features and advantages of the present disclosure will become more apparent upon reading the following specification in conjunction with the accompanying description, claims and drawings.

### BRIEF DESCRIPTION OF THE DRAWINGS

The accompanying Figures, which are incorporated in and constitute a part of this specification, illustrate several aspects described below.

FIGS. 1A-1D depict a conceptual diagram of modeling immune response in health and disease. (1A) Immune response as dynamically regulated in health (left) and dysfunctional in chronic conditions (right). (1B) Alternative block diagram showing an embodiment of the claimed invention. (1C) Alternative block diagram with macrophages as the system or plant that is being controlled. (1D) Identification, validation and prediction of inflammatory response as a three-step process consisting of design of (panel 1) an engineering model structure and fit of model parameters, (panel 2) comparison of predicted and experimental results, and (panel 3) use of the predictive model to design input strategies to obtain a desired response.

FIGS. 2A-2D show that RAW264.7 macrophages transiently express iNOS in response to constant or repeated LPS stimulation. (2A) Representative Western blot for iNOS (140 kDa) and α-tubulin (55 kDa) after LPS treatment. (2B) ICC quantification matches Western blot analysis of transient iNOS expression in response to a single administration of LPS. (2C) Dynamics of iNOS expression are not modulated in response to multiple administrations of LPS or (2D) after 24 hours in basal medium before LPS re-stimulation (mean±SEM, N=16 at 0, 24, 48, 72 hrs; gray curves; interpolation±RMS CV error).

FIGS. 3A-3L show single input/single output (SISO) LPS/iNOS ARX model, controller design, and experimental MPC testing. (3A) Identified ARX model of macrophage iNOS response to LPS has a characteristic step response that follows the biologically quantified trajectory. Control system design identifies input strategy (dashed line) for a step reference that elicits a gradual increase in plant response (blue stems) using a (3B) PI or (3C) LQG controller. Experimental implementation using cultured Raw 264.7 macrophages and (3D, 3G) PI controller-, (3E, 3H) LQG controller-, or (3F, 3I) a combination of designed LPS input schema (dashed line) modulates temporal iNOS expression (red curves, mean±SEM, N=16; interpolated curve±RMS CV error) but does not reach the unit reference nor sustain 72 hr activity. (3J-3L) show the PI controller (3J), LQG controller (3K), and combination (3L) with the effects of decay.

FIGS. 4A-4C show that orthogonal stimuli maintain or magnify iNOS expression. (4A) Signaling diagram for LPS and IFN-γ (created with BioRender). (2B) 24 hrs of LPS treatment and delayed subsequent IFN-γ (dashed lines) treatment modulates iNOS expression (gray curves, mean±SEM, N=16; interpolated curve±RMS CV error) even at 72 hr time point. (2C) 24 hrs of LPS treatment and immediately subsequent IFN-γ (dashed lines) treatment modulates iNOS expression (gray curves, mean±SEM, N=16; interpolated curve±RMS CV error) even at 72 hr time point.

FIGS. 5A-5D show that Raw 264.7 macrophages are markedly affected by activation state-dependent hysteresis which can be overcome using multiple pro-inflammatory inputs. (5A) LPS and IFN-γ added simultaneously cause time dependent supra-additive expression of iNOS (color and text display condition mean; N=4). (5B) Pretreating macrophages with IL-4 for 24 hours prior to LPS stimulation reduces the magnitude of pro-inflammatory polarization, measured by iNOS expression normalized by DAPI (shade represents mean, SEM displayed numerically, N=4). (5C) Combining IFN-γ with LPS recovers iNOS expression, dose-dependently overcoming the hysteretic effect (shade represents mean, SEM displayed numerically, N=4). (5D) Interpolated attenuation factor fit error.

FIG. 6 shows an exemplary linear and nonlinear global plant. Detailed diagram of multiple input plant model implemented in control system (as shown in FIG. 1C). System predicted inputs u1 (LPS) and u2 (IFN-γ) are fed into respective identified SISO ARX models and supra-additive interaction term λ elements. Terms multiplied by weighting coefficients c (defined by multiple regression estimation) prior to summation (Σ) and hysteresis-dependent attenuation (γ).

FIGS. 7A-7G show that open-loop control of pro-inflammatory macrophage activity is experimentally achieved using a nested multiple regression. (7A) Raw264.7 macrophage temporal response to 1 μg/ml LPS and 100 ng/ml IFN-γ. First generation hysteresis-free nested regression model given temporally variable input u1 and u2 (7B and 7C) approaches step reference after minor overshoot. First generation nested model including hysteresis term predicts inputs given in (7D) will achieve the desired set point (7E, bottom light gray curve). A non-hysteretic model given inputs in (7D) will overshoot the reference (7E, top dark gray curve). Experimental delivery of designed inputs (7D) reflects predicted control output (7E) for both hysteretic (7F, bottom light gray curve, mean±SEM, N=16; interpolated curve±RMS CV error) and non-hysteretic (7F, top dark gray curve, mean±SEM, N=16; interpolated curve±RMS CV error) Raw264.7 macrophage cultures. (7G) shows designed inputs in the second generation model for both hysteretic (bottom light gray curve) and non-hysteretic (top dark gray curve).

FIG. 8 shows Western blot quantification of in vitro Raw264.7 macrophage iNOS protein expression after treatment with LPS, IL-4, or control media shows iNOS peaks at 24 hrs of LPS treatment but is not expressed in IL-4 conditions (n=2; mean±min/max range).

FIG. 9 shows the choice of orthogonal input.

FIG. 10 shows that Raw 264.7 macrophage temporally dynamic response to 100 ng/ml IFN-γ alone is distinct from the LPS response but is also not sustained.

FIG. 11 shows Arg1 expression for hysteresis M2 polarization validation.

### DETAILED DESCRIPTION OF THE DISCLOSURE

As specified in the Background Section, there is a great need in the art to identify technologies for restoring proper regulation of the immune response and use this understanding to develop novel methods and systems for exogenously controlling immune cells in order to dynamically and predictively drive the immune response through pro-inflammatory activity to anti-inflammatory activity, mimicking the immune system's natural progression through these states. The present disclosure satisfies this and other needs. Embodiments of the present disclosure relate generally to methods and systems for dynamic predictive modeling and control of inflammation and the immune response, and more specifically to methods and systems for predictive modeling and control of the inflammatory state of immune cells via temporally regulated immune-modulating stimuli.

### Definitions

To facilitate an understanding of the principles and features of the various embodiments of the disclosure, various illustrative embodiments are explained below. Although exemplary embodiments of the disclosure are explained in detail, it is to be understood that other embodiments are contemplated. Accordingly, it is not intended that the disclosure is limited in its scope to the details of construction and arrangement of components set forth in the following description or examples. The disclosure is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present disclosure as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claimed disclosure or to imply that certain features are critical, essential, or even important to the structure or function of the claimed disclosure. Rather, these terms are merely intended to highlight alternative or additional features that may or may not be utilized in a particular embodiment of the present disclosure. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

As used herein, the term “subject” or “patient” refers to mammals and includes, without limitation, human and veterinary animals. In a preferred embodiment, the subject is human.

A “disease” is a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “therapeutic” as used herein means a treatment and/or prophylaxis. A therapeutic effect is obtained by suppression, diminution, remission, or eradication of a disease state.

As used herein the term “therapeutically effective” applied to dose or amount refers to that quantity of a compound or pharmaceutical composition that when administered to a subject for treating (e.g., preventing or ameliorating) a state, disorder or condition, is sufficient to effect such treatment. The “therapeutically effective amount” will vary depending on the compound or bacteria or analogues administered as well as the disease and its severity and the age, weight, physical condition and responsiveness of the mammal to be treated.

As used herein, the term “immune response” includes myeloid cells, such as macrophages, microglia, eosinophils, mast cells, basophils, and granulocytes. Exemplary immune responses include macrophage polarization, e.g., including expression of classical markers of M1 or M2 phenotypes, cytokine production. The term “immune response” also includes T-cell mediated and/or B-cell mediated immune responses, e.g., cytokine production and cellular cytotoxicity, and B cell responses, e.g., antibody production. In addition, the term “immune response” includes immune responses that are indirectly affected by T cell activation, e.g., antibody production (humoral responses) and activation of cytokine responsive cells, e.g., macrophages. Immune cells involved in the immune response include lymphocytes, such as B cells and T cells (CD4+, CD8+, Th1 and Th2 cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells.

In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

In accordance with the present disclosure there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

### Methods of the Disclosure

In one aspect, the disclosure provides a method for dynamic real-time modeling and/or control of an inflammatory response in an immune cell, comprising: providing a fluid chamber comprising at least one inlet, at least one outlet, and the immune cell; delivering a first stimulus through the inlet via a controller, the controller in fluid communication with the fluid chamber, wherein the stimulus elicits a change in an inflammatory state of the immune cell; and detecting the change in the inflammatory state of the immune cell via a detector, the detector in fluid communication with the fluid chamber, wherein the controller is configured to deliver a second stimulus based on the change in the inflammatory state of the immune cell in order to model and/or control the inflammatory response of the immune cell, wherein the detector is configured to generate input and/or output data indicative of the change in the inflammatory state of the immune cell, and wherein the change in the inflammatory state of the immune cell to each of the first stimulus and second stimulus is predicted by the steps of: fitting a black box engineering model to the input and/or output data obtained by stimulating cells within the chamber; and selecting a best fitting black box engineering model based on the input and/or output data and applying the best fitting black box engineering model to future input and/or output data. Non-limiting exemplary black box engineering models are described in Lennart Ljung, ed., System Identification: Theory for the User, 2^{nd} Edition (1999).

In another aspect, the disclosure provides a method of treating a disease or condition in a subject in need thereof caused by an aberrant inflammatory response comprising: monitoring and/or controlling in real time the aberrant inflammatory response in an immune cell, comprising: providing a fluid chamber comprising at least one inlet, at least one outlet, and the immune cell; delivering a first stimulus through the inlet via a controller, the controller in fluid communication with the fluid chamber, wherein the stimulus elicits a change in an inflammatory state of the immune cell; and detecting the change in the inflammatory state of the immune cell via a detector, the detector in fluid communication with the fluid chamber, wherein the controller is configured to deliver a second stimulus based on the change in the inflammatory state of the immune cell in order to model and/or control the inflammatory response of the immune cell, wherein the detector is configured to generate input and/or output data indicative of the change in the inflammatory state of the immune cell, and wherein the change in the inflammatory state of the immune cell to each of the first stimulus and second stimulus is predicted by the steps of: fitting a black box engineering model to the input and/or output data obtained by stimulating cells within the chamber; and selecting a best fitting black box engineering model based on the input and/or output data and applying the best fitting black box engineering model to future input and/or output data, and wherein the first and/or second stimulus is administered to the subject in order to control the aberrant inflammatory response thereby treating the disease or condition.

In any of the foregoing aspects, the method can further comprise one or more of the following embodiments. Each combination is specifically contemplated herein.

In any of the embodiments disclosed herein, the fluid chamber can be a cell culture chamber, a cell culture well, or a microfluidic chamber.

In any of the embodiments disclosed herein, the immune cell can comprise a microglial cell, an astrocyte, a macrophage, a B cell, a T cell, a natural killer (NK) cell, and a leukocyte. In any of the embodiments disclosed herein, the immune cell can comprise at least one cell selected from the following types of cells: a microglial cell, an astrocyte, a macrophage, a B cell, a T cell, a natural killer (NK) cell, and a leukocyte. In some embodiments, the immune cell can be obtained from the subject having the disease or condition. In some embodiments, the immune cell can comprise a microglial cell, a macrophage, or combinations thereof. In some embodiments, different types of immune cells can be utilized.

In any of the embodiments disclosed herein, the first stimulus and the second stimulus can each comprise at least one immune-modulating molecule. In any of the embodiments disclosed herein, the at least one immune-modulating molecule can be pro-inflammatory or anti-inflammatory. In any of the embodiments disclosed herein, the at least one immune-modulating molecule can comprise an antigen, a cytokine, a growth factor, a sphingolipid, a complement factor, an immunomodulatory small molecule, an intracellular signaling inhibitor, an activator of pro-inflammatory or anti-inflammatory pathways, a cytokine inhibitor, and combinations thereof.

In any of the embodiments disclosed herein, a first immune-modulating molecule can be administered at the same time as a second immune-modulating molecule. In any of the embodiments disclosed herein, a first immune-modulating molecule can be administered before a second immune-modulating molecule. In any of the embodiments disclosed herein, the first immune-modulating molecule can be administered between five minutes and 24 hours before the second immune-modulating molecule.

In any of the embodiments disclosed herein, the first immune-modulating molecule can be different from a second immune-modulating molecule. In any of the embodiments disclosed herein, the first immune-modulating molecule can be the same as a second immune-modulating molecule.

In any of the embodiments disclosed herein, the dosage or concentration of one or both of the first immune-modulating molecule and the second immune-modulating molecule can be continuously varied.

In any of the embodiments disclosed herein, one or both of the first immune-modulating molecule and the second immune-modulating molecule can stimulate the immune system. In any of the embodiments disclosed herein, one or both of the first immune-modulating molecule and the second immune-modulating molecule can suppress the immune system.

In any of the embodiments disclosed herein, the first stimulus can cause the immune cell to change from a pro-inflammatory state to an anti-inflammatory state. In any of the embodiments disclosed herein, the first stimulus can cause the immune cell to change from a quiescent state or homeostatic state to a pro-inflammatory state. In any of the embodiments disclosed herein, the first stimulus can cause the immune cell to change from an anti-inflammatory state to a pro-inflammatory state.

In any of the embodiments disclosed herein, the change in the inflammatory state of the immune cell can be detected by measuring a marker characteristic of the inflammatory state. In any of the embodiments disclosed herein, a marker characteristic of the pro-inflammatory state can comprise iNOS, SOCS3, TLR4, TLR2, IL-1R, MHCII, CD68, CD80, CD86, TLR2, TNFα, IL1α, ITAM1, IL1β, HIF1α, IL-12b, KCna3, GFAP, CLEC7a, Vimentin, CD69, CD27, CD45, CD44, and CCR7.

In any of the embodiments disclosed herein, a marker characteristic of the pro-inflammatory state can comprise iNOS, SOCS3, TLR4, TLR2, IL-1R, MHCII, CD68, CD80, and CD86 and the immune cell can be a macrophage. In any of the embodiments disclosed herein, a marker characteristic of the pro-inflammatory state can comprise TLR2, TNFα, IL1α, ITAM1, iNOS, IL1β, HIF1α, IL-12b, and KCna3 and the immune cell can be a microglial cell. In any of the embodiments disclosed herein, a marker characteristic of the pro-inflammatory state can comprise GFAP, CLEC7a, and Vimentin and the immune cell can be an astrocyte. In any of the embodiments disclosed herein, a marker characteristic of the pro-inflammatory state can comprise CD69, CD27, CD45, CD44, and CCR7 and the immune cell can be a T cell.

In any of the embodiments disclosed herein, a marker characteristic of the anti-inflammatory state or homeostatic state can comprise CD163, MHCII, SR, CD206, CD200R, TGM2, DecoyR, IL-1R, Ym1/2, Fizz1, Arg1, CD86, TLR1, TLR8, VEGF, Arg1, APOE, TIMP2, IGF1, DPP6, P2Rγ12, TMEM119, BIN1, PTGS1, and CD62. In any of the embodiments disclosed herein, a marker characteristic of the anti-inflammatory state or homeostatic state can comprise CD163, MHCII, SR, CD206, CD200R, TGM2, DecoyR, IL-1R, Ym1/2, Fizz1, Arg1, CD86, TLR1, TLR8, and VEGF and the immune cell can be a macrophage. In any of the embodiments disclosed herein, a marker characteristic of the anti-inflammatory state or homeostatic state can comprise Arg1, APOE, TIMP2, IGF1, DPP6, P2Rγ12, TMEM119, BIN1, PTGS1, and CD62 and the immune cell can be a microglial cell.

In any of the embodiments disclosed herein, the second stimulus can be provided to achieve or maintain the anti-inflammatory state or quiescent state of the immune cell. In any of the embodiments disclosed herein, the second stimulus can be provided to suppress the inflammatory response at a desired interval. In any of the embodiments disclosed herein, the second stimulus can comprise at least one immune-modulating molecule. In any of the embodiments disclosed herein, the at least one immune-modulating molecule can comprise an antigen, a cytokine, a growth factor, a sphingolipid, a complement factor, an immunomodulatory small molecule, an intracellular signaling inhibitor, an activator of pro-inflammatory or anti-inflammatory pathways, a cytokine inhibitor, and combinations thereof.

In any of the embodiments disclosed herein, the system can be an open-loop system. In an open-loop system, the sequence of stimuli can be pre-determined based on the predictive dynamic model. In an open-loop system, the detector can measure a detectable marker of the inflammatory state of the immune cell, such as a labeled marker (e.g., a fluorescently labeled marker, a luminescent marker, a marker that is labeled with a marker detectable at a certain wavelength, a colorimetric marker, and a radiolabeled marker). An open-loop system can also enable endpoint measurement such as for example and not limitation, a Western blot, ELISA, RNA sequencing, qPCR, qRTPCR, and mass spectrometry. An open-loop system can also measure a detectable output comprising colorimetric, luminescent, radioactive or fluorescent reporters of immune marker expression or level. The immune marker can comprise a cell surface marker or a secreted factor and measured at the protein or transcript level.

In any of the embodiments disclosed herein, the system can be a closed-loop system. In a closed-loop system, the detector can be configured to detect the change in the inflammatory state of the immune cell in real time. This detection in real time can enable the quantification of the change in inflammatory state and active updating of the timing, concentration, dosage, and/or duration of one or both of the first stimulus and the second stimulus via the controller. The change in inflammatory state of the immune cell can be accounted for and adjusted in real time as the immune response proceeds. In a closed-loop system, the detector can be configured to detect colorimetric, luminescent, radioactive or fluorescent output indicative of the change in the inflammatory state of the immune cell, and the controller can be configured to increase or decrease the amount of the first stimulus or second stimulus in response to the input/output data obtained from the detector. In a closed-loop system, the colorimetric, luminescent, radioactive or fluorescent output can comprise colorimetric, luminescent, radioactive or fluorescent reporters of immune marker expression or level. The detector can also be configured to measure a detectable marker of the inflammatory state of the immune cell, such as a labeled marker (e.g., a fluorescently labeled marker, a luminescent marker, a marker that is labeled with a marker detectable at a certain wavelength, a colorimetric marker, and a radiolabeled marker). The detector can also be configured to measure a detectable output comprising colorimetric, luminescent, radioactive or fluorescent reporters of immune marker expression or level.

In any of the embodiments disclosed herein, the detector can further be configured to detect immune marker expression or level. In any of the embodiments disclosed herein, the immune marker can comprise a cell surface marker or a secreted factor. In any of the embodiments disclosed herein, the immune marker can be labeled with a detectable marker comprising a fluorescent marker, a bioluminescent marker, a colorimetric marker, and a radioactive marker. In any of the embodiments disclosed herein, the immune marker can comprise a cell surface marker or a secreted factor.

In any of the embodiments disclosed herein, the fluid chamber further can comprise a fluid medium suitable for growth and/or expansion of the immune cell.

In any of the embodiments disclosed herein, the black box engineering model used to predict the change in inflammatory state of the immune cell can be include or be constructed from a finite impulse response (FIR) model, an autroregressive with exogenous input terms (ARX) model, an autoregressive-moving-average (ARMA) model. The black box model may be constructed from an orthogonal basis function, such as a Laguerre series basis function. These functions may be combined in either linear or non-linear configurations.

In another aspect, the disclosure provides a method of treating a disease or condition in a subject in need thereof caused by an aberrant inflammatory response comprising: administering a first stimulus to the subject, wherein the stimulus elicits a change in an inflammatory state of the subject's immune cells; obtaining a biological sample from the subject; detecting the change in the inflammatory state via a detector; delivering a second stimulus based on the change in the inflammatory state of the immune cell in order to model and/or control the inflammatory response of the immune cells, wherein the detector is configured to generate input and/or output data indicative of the change in the inflammatory state of the immune cells, wherein the change in the inflammatory state of the immune cells to each of the first stimulus and second stimulus is predicted by the steps of: fitting a black box engineering model to the input and/or output data obtained by stimulating the subject's immune cells; and selecting the best fitting black box engineering model based on the input and/or output data and applying the best fitting black box engineering model to future input and/or output data, and wherein the first and/or second stimulus is administered to the subject in order to control the aberrant inflammatory response thereby treating the disease or condition.

In any of the foregoing aspects, the method can further comprise one or more of the following embodiments. Each combination is specifically contemplated herein.

In any of the embodiments disclosed herein, the disease or condition caused by the aberrant immune response can comprise an inflammatory disease, such as Alzheimer's disease, Parkinson's disease, frontotemporal dementia, schizophrenia, traumatic brain injury, rheumatoid arthritis, inflammatory bowel disease, chronic obstructive pulmonary disease, and diabetic ulcers.

In any of the embodiments disclosed herein, the biological sample comprises a biological fluid or tissue. In any of the embodiments disclosed herein, the biological fluid is selected from the group consisting of blood, serum, plasma, urine, saliva, tears, mucus, lymph, interstitial fluid, cerebrospinal fluid, pus, breast milk, and amniotic fluid.

In any of the embodiments disclosed herein, the immune cell can comprise a microglial cell, an astrocyte, a macrophage, a B cell, a T cell, a natural killer (NK) cell, and a leukocyte. In some embodiments, the immune cell can be obtained from the subject having the disease or condition. In some embodiments, the immune cell can comprise a microglial cell and a macrophage.

In any of the embodiments disclosed herein, the first stimulus and the second stimulus can each comprise at least one immune-modulating molecule. In any of the embodiments disclosed herein, the at least one immune-modulating molecule can be pro-inflammatory or anti-inflammatory. In any of the embodiments disclosed herein, the at least one immune-modulating molecule can comprise an antigen, a cytokine, a growth factor, a sphingolipid, a complement factor, an immunomodulatory small molecule, an intracellular signaling inhibitor, an activator of pro-inflammatory or anti-inflammatory pathways, a cytokine inhibitor, and combinations thereof.

In any of the embodiments disclosed herein, a first immune-modulating molecule can be administered at the same time as a second immune-modulating molecule. In any of the embodiments disclosed herein, a first immune-modulating molecule can be administered before a second immune-modulating molecule. In any of the embodiments disclosed herein, the first immune-modulating molecule can be administered between five minutes and 24 hours before the second immune-modulating molecule.

In any of the embodiments disclosed herein, the first immune-modulating molecule can be different from a second immune-modulating molecule. In any of the embodiments disclosed herein, the first immune-modulating molecule can be the same as a second immune-modulating molecule.

In any of the embodiments disclosed herein, the dosage or concentration of one or both of the first immune-modulating molecule and the second immune-modulating molecule can be continuously varied.

In any of the embodiments disclosed herein, one or both of the first immune-modulating molecule and the second immune-modulating molecule can stimulate the immune system. In any of the embodiments disclosed herein, one or both of the first immune-modulating molecule and the second immune-modulating molecule can suppress the immune system.

In any of the embodiments disclosed herein, the first stimulus can cause the immune cell to change from a pro-inflammatory state to an anti-inflammatory state. In any of the embodiments disclosed herein, the first stimulus can cause the immune cell to change from a quiescent state to a pro-inflammatory state. In any of the embodiments disclosed herein, the first stimulus can cause the immune cell to change from a homeostatic state to a pro-inflammatory state. In any of the embodiments disclosed herein, the first stimulus can cause the immune cell to change from an anti-inflammatory state to a pro-inflammatory state.

In any of the embodiments disclosed herein, the change in the inflammatory state of the immune cell can be detected by measuring a marker characteristic of the inflammatory state. In any of the embodiments disclosed herein, a marker characteristic of the pro-inflammatory state can comprise iNOS, SOCS3, TLR4, TLR2, IL-1R, MHCII, CD68, CD80, CD86, TLR2, TNFα, IL1α, ITAM1, IL1β, HIF1α, IL-12b, KCna3, GFAP, CLEC7a, Vimentin, CD69, CD27, CD45, CD44, and CCR7.

In any of the embodiments disclosed herein, a marker characteristic of the pro-inflammatory state can comprise iNOS, SOCS3, TLR4, TLR2, IL-1R, MHCII, CD68, CD80, and CD86 and the immune cell can be a macrophage. In any of the embodiments disclosed herein, a marker characteristic of the pro-inflammatory state can comprise TLR2, TNFα, IL1α, ITAM1, iNOS, IL1β, HIF1α, IL-12b, and KCna3 and the immune cell can be a microglial cell. In any of the embodiments disclosed herein, a marker characteristic of the pro-inflammatory state can comprise GFAP, CLEC7a, and Vimentin and the immune cell can be an astrocyte. In any of the embodiments disclosed herein, a marker characteristic of the pro-inflammatory state can comprise CD69, CD27, CD45, CD44, and CCR7 and the immune cell can be a T cell.

In any of the embodiments disclosed herein, a marker characteristic of the anti-inflammatory state or homeostatic state can comprise CD163, MHCII, SR, CD206, CD200R, TGM2, DecoyR, IL-1R, Ym1/2, Fizz1, Arg1, CD86, TLR1, TLR8, VEGF, Arg1, APOE, TIMP2, IGF1, DPP6, P2Rγ12, TMEM119, BIN1, PTGS1, and CD62. In any of the embodiments disclosed herein, a marker characteristic of the anti-inflammatory state or homeostatic state can comprise CD163, MHCII, SR, CD206, CD200R, TGM2, DecoyR, IL-1R, Ym1/2, Fizz1, Arg1, CD86, TLR1, TLR8, and VEGF and the immune cell can be a macrophage. In any of the embodiments disclosed herein, a marker characteristic of the anti-inflammatory state or homeostatic state can comprise Arg1, APOE, TIMP2, IGF1, DPP6, P2Rγ12, TMEM119, BIN1, PTGS1, and CD62 and the immune cell can be a microglial cell.

In any of the embodiments disclosed herein, the second stimulus can be provided to achieve or maintain the anti-inflammatory state or quiescent state of the immune cell. In any of the embodiments disclosed herein, the second stimulus can be provided to suppress the inflammatory response at a desired interval. In any of the embodiments disclosed herein, the second stimulus can comprise at least one immune-modulating molecule. In any of the embodiments disclosed herein, the at least one immune-modulating molecule can comprise an antigen, a cytokine, a growth factor, a sphingolipid, a complement factor, an immunomodulatory small molecule, an intracellular signaling inhibitor, an activator of pro-inflammatory or anti-inflammatory pathways, a cytokine inhibitor, and combinations thereof.

In any of the embodiments disclosed herein, the detector can be configured to detect the change in the inflammatory state of the immune cell in real time. This detection in real time can enable the quantification of the change in inflammatory state and active updating of the timing, concentration, dosage, and/or duration of the administration of one or both of the first stimulus and the second stimulus to the subject. The change in inflammatory state of the immune cell can be accounted for and adjusted in real time as the immune response proceeds. In a closed-loop system, the detector can be configured to detect colorimetric, luminescent, radioactive or fluorescent output indicative of the change in the inflammatory state of the immune cell, and the controller can be configured to increase or decrease the amount of the first stimulus or second stimulus in response to the input/output data obtained from the detector. In a closed-loop system, the colorimetric, luminescent, radioactive or fluorescent output can comprise colorimetric, luminescent, radioactive or fluorescent reporters of immune marker expression or level.

In any of the embodiments disclosed herein, the detector can further be configured to detect immune marker expression or level. In any of the embodiments disclosed herein, the immune marker can comprise a cell surface marker or a secreted factor. In any of the embodiments disclosed herein, the immune marker can be labeled with a detectable marker comprising a fluorescent marker, a bioluminescent marker, a colorimetric marker, and a radioactive marker. In any of the embodiments disclosed herein, the immune marker can comprise a cell surface marker or a secreted factor.

In any of the embodiments disclosed herein, a second therapeutic suitable to treat the disease or condition can be administered at a therapeutically effective amount. The second therapeutic can be administered before, simultaneously with, or after one or both of the first stimulus and the second stimulus.

In any of the embodiments disclosed herein, the black box engineering model used to predict the change in inflammatory state of the immune cell can be include or be constructed from a finite impulse response (FIR) model, an autroregressive with exogenous input terms (ARX) model, an autoregressive-moving-average (ARMA) model. The black box model may be constructed from an orthogonal basis function, such as a Laguerre series basis function. These functions may be combined in either linear or non-linear configurations.

It is contemplated that when used to treat various diseases, the methods of the present disclosure can be combined with other therapeutic agents suitable for the same or similar diseases. When co-administered with a therapeutic agent, the embodiment of the disclosure and the second therapeutic agent may be simultaneously or sequentially (in any order). Suitable therapeutically effective dosages for the therapeutic agent may be lowered due to additive action or synergy. As a non-limiting example, the disclosure can be combined with other therapies that block inflammation (e.g., corticosteroids or via blockage of ILL INFNα/β, IL6, TNFα, IL13, IL23, etc.) or that modulate immune responses.

Administration of the compounds and compositions in the methods of the disclosure can be accomplished by any method known in the art. Non-limiting examples of useful routes of delivery include oral, rectal, fecal (by enema), and via naso/oro-gastric gavage, as well as parenteral, intraperitoneal, intradermal, transdermal, intrathecal, nasal, and intracheal administration. The active agent may be systemic after administration or may be localized by the use of regional administration, intramural administration, or use of an implant that acts to retain the active dose at the site of implantation.

### Systems of the Disclosure

In another aspect, the disclosure provides a system for dynamic real-time modeling and/or control of an inflammatory response in an immune cell, comprising: a fluid chamber comprising at least one inlet, at least one outlet, and the immune cell; a controller in fluid communication with the fluid chamber configured to deliver a first stimulus through the inlet, wherein the stimulus elicits a change in the inflammatory state of the immune cell; and a detector in fluid communication with the fluid chamber configured to detect the change in the inflammatory state of the immune cell, wherein the controller is further configured to deliver a second stimulus based on the change in the inflammatory state of the immune cell in order to model and/or control the inflammatory response of the immune cell, wherein the detector is configured to generate input and/or output data indicative of the change in the inflammatory state of the immune cell, and wherein the change in the inflammatory state of the immune cell to each of the first stimulus and second stimulus is predicted by the steps of: fitting a black box engineering model to the input and/or output data obtained by stimulating cells within the chamber; and selecting a best fitting black box engineering model based on the input/output data and applying the best fitting black box engineering model to future input and/or output data.

In another aspect, the disclosure provides a system for treating a disease or condition in a subject in need thereof caused by an aberrant inflammatory response comprising: monitoring and/or controlling in real time the aberrant inflammatory response in an immune cell, comprising: providing a fluid chamber comprising at least one inlet, at least one outlet, and the immune cell; delivering a first stimulus through the inlet via a controller, the controller in fluid communication with the fluid chamber, wherein the stimulus elicits a change in the inflammatory state of the immune cell; and detecting the change in the inflammatory state of the immune cell via a detector, the detector in fluid communication with the fluid chamber, wherein the controller is configured to deliver a second stimulus based on the change in the inflammatory state of the immune cell in order to model and/or control the inflammatory response of the immune cell, wherein the detector is configured to generate input and/or output data indicative of the change in the inflammatory state of the immune cell, wherein the change in the inflammatory state of the immune cell to each of the first stimulus and second stimulus is predicted by the steps of: fitting a black box engineering model to the input and/or output data obtained by stimulating cells within the chamber; and selecting a best fitting black box engineering model based on the input and/or output data and applying the best fitting black box engineering model to future input and/or output data, and wherein the first and/or second stimulus is administered to the subject in order to control the aberrant inflammatory response thereby treating the disease or condition.

In any of the foregoing aspects, the system can further comprise one or more of the following embodiments. Each combination is specifically contemplated herein.

In any of the embodiments disclosed herein, the fluid chamber can be a cell culture chamber, a cell culture well, or a microfluidic chamber.

In any of the embodiments disclosed herein, the immune cell can comprise a microglial cell, an astrocyte, a macrophage, a B cell, a T cell, a natural killer (NK) cell, and a leukocyte. In any of the embodiments disclosed herein, the immune cell can comprise at least one cell selected from the following types of cells: a microglial cell, an astrocyte, a macrophage, a B cell, a T cell, a natural killer (NK) cell, and a leukocyte. In some embodiments, the immune cell can be obtained from the subject having the disease or condition. In some embodiments, the immune cell can comprise a microglial cell, a macrophage, or combinations thereof. In some embodiments, different types of immune cells can be utilized.

In any of the embodiments disclosed herein, the first stimulus and the second stimulus can each comprise at least one immune-modulating molecule. In any of the embodiments disclosed herein, the at least one immune-modulating molecule can be pro-inflammatory or anti-inflammatory. In any of the embodiments disclosed herein, the at least one immune-modulating molecule can comprise an antigen, a cytokine, a growth factor, a sphingolipid, a complement factor, an immunomodulatory small molecule, an intracellular signaling inhibitor, an activator of pro-inflammatory or anti-inflammatory pathways, a cytokine inhibitor, and combinations thereof.

In any of the embodiments disclosed herein, a first immune-modulating molecule can be administered at the same time as a second immune-modulating molecule. In any of the embodiments disclosed herein, a first immune-modulating molecule can be administered before a second immune-modulating molecule. In any of the embodiments disclosed herein, the first immune-modulating molecule can be administered between five minutes and 24 hours before the second immune-modulating molecule.

In any of the embodiments disclosed herein, the first immune-modulating molecule can be different from a second immune-modulating molecule. In any of the embodiments disclosed herein, the first immune-modulating molecule can be the same as a second immune-modulating molecule.

In any of the embodiments disclosed herein, the dosage or concentration of one or both of the first immune-modulating molecule and the second immune-modulating molecule can be continuously varied.

In any of the embodiments disclosed herein, one or both of the first immune-modulating molecule and the second immune-modulating molecule can stimulate the immune system. In any of the embodiments disclosed herein, one or both of the first immune-modulating molecule and the second immune-modulating molecule can suppress the immune system.

In any of the embodiments disclosed herein, the first stimulus can cause the immune cell to change from a pro-inflammatory state to an anti-inflammatory state. In any of the embodiments disclosed herein, the first stimulus can cause the immune cell to change from a quiescent state to a pro-inflammatory state. In any of the embodiments disclosed herein, the first stimulus can cause the immune cell to change from a homeostatic state to a pro-inflammatory state. In any of the embodiments disclosed herein, the first stimulus can cause the immune cell to change from an anti-inflammatory state to a pro-inflammatory state.

In any of the embodiments disclosed herein, the change in the inflammatory state of the immune cell can be detected by measuring a marker characteristic of the inflammatory state. In any of the embodiments disclosed herein, a marker characteristic of the pro-inflammatory state can comprise iNOS, SOCS3, TLR4, TLR2, IL-1R, MHCII, CD68, CD80, CD86, TLR2, TNFα, IL1α, ITAM1, IL1β, HIF1α, IL-12b, KCna3, GFAP, CLEC7a, Vimentin, CD69, CD27, CD45, CD44, and CCR7.

In any of the embodiments disclosed herein, a marker characteristic of the pro-inflammatory state can comprise iNOS, SOCS3, TLR4, TLR2, IL-1R, MHCII, CD68, CD80, and CD86 and the immune cell can be a macrophage. In any of the embodiments disclosed herein, a marker characteristic of the pro-inflammatory state can comprise TLR2, TNFα, IL1α, ITAM1, iNOS, IL1β, HIF1α, IL-12b, and KCna3 and the immune cell can be a microglial cell. In any of the embodiments disclosed herein, a marker characteristic of the pro-inflammatory state can comprise GFAP, CLEC7a, and Vimentin and the immune cell can be an astrocyte. In any of the embodiments disclosed herein, a marker characteristic of the pro-inflammatory state can comprise CD69, CD27, CD45, CD44, and CCR7 and the immune cell can be a T cell.

In any of the embodiments disclosed herein, a marker characteristic of the anti-inflammatory state or homeostatic state can comprise CD163, MHCII, SR, CD206, CD200R, TGM2, DecoyR, IL-1R, Ym1/2, Fizz1, Arg1, CD86, TLR1, TLR8, VEGF, Arg1, APOE, TIMP2, IGF1, DPP6, P2Rγ12, TMEM119, BIN1, PTGS1, and CD62. In any of the embodiments disclosed herein, a marker characteristic of the anti-inflammatory state or homeostatic state can comprise CD163, MHCII, SR, CD206, CD200R, TGM2, DecoyR, IL-1R, Ym1/2, Fizz1, Arg1, CD86, TLR1, TLR8, and VEGF and the immune cell can be a macrophage. In any of the embodiments disclosed herein, a marker characteristic of the anti-inflammatory state or homeostatic state can comprise Arg1, APOE, TIMP2, IGF1, DPP6, P2Rγ12, TMEM119, BIN1, PTGS1, and CD62 and the immune cell can be a microglial cell.

In any of the embodiments disclosed herein, the second stimulus can be provided to achieve or maintain the anti-inflammatory state or quiescent state of the immune cell. In any of the embodiments disclosed herein, the second stimulus can be provided to suppress the inflammatory response at a desired interval. In any of the embodiments disclosed herein, the second stimulus can comprise at least one immune-modulating molecule. In any of the embodiments disclosed herein, the at least one immune-modulating molecule can comprise an antigen, a cytokine, a growth factor, a sphingolipid, a complement factor, an immunomodulatory small molecule, an intracellular signaling inhibitor, an activator of pro-inflammatory or anti-inflammatory pathways, a cytokine inhibitor, and combinations thereof.

In any of the embodiments disclosed herein, the system can be an open-loop system. In an open-loop system, the sequence of stimuli can be pre-determined based on the predictive dynamic model. In an open-loop system, the detector can measure a detectable marker of the inflammatory state of the immune cell, such as a labeled marker (e.g., a fluorescently labeled marker, a luminescent marker, a marker that is labeled with a marker detectable at a certain wavelength, a colorimetric marker, and a radiolabeled marker). An open-loop system can also enable endpoint measurement such as for example and not limitation, a Western blot, ELISA, RNA sequencing, qPCR, qRTPCR, and mass spectrometry. An open-loop system can also measure a detectable output comprising colorimetric, luminescent, radioactive or fluorescent reporters of immune marker expression or level. The immune marker can comprise a cell surface marker or a secreted factor.

In any of the embodiments disclosed herein, the system can be a closed-loop system. In a closed-loop system, the detector can be configured to detect the change in the inflammatory state of the immune cell in real time. This detection in real time can enable the quantification of the change in inflammatory state and active updating of the timing, concentration, dosage, and/or duration of one or both of the first stimulus and the second stimulus via the controller. The change in inflammatory state of the immune cell can be accounted for and adjusted in real time as the immune response proceeds. In a closed-loop system, the detector can be configured to detect colorimetric, luminescent, radioactive or fluorescent output indicative of the change in the inflammatory state of the immune cell, and the controller can be configured to increase or decrease the amount of the first stimulus or second stimulus in response to the input/output data obtained from the detector. In a closed-loop system, the colorimetric, luminescent, radioactive or fluorescent output can comprise colorimetric, luminescent, radioactive or fluorescent reporters of immune marker expression or level. The detector can also be configured to measure a detectable marker of the inflammatory state of the immune cell, such as a labeled marker (e.g., a fluorescently labeled marker, a luminescent marker, a marker that is labeled with a marker detectable at a certain wavelength, a colorimetric marker, and a radiolabeled marker). The detector can also be configured to measure a detectable output comprising colorimetric, luminescent, radioactive or fluorescent reporters of immune marker expression or level.

In any of the embodiments disclosed herein, the detector can be configured to detect immune marker expression or level. In any of the embodiments disclosed herein, the immune marker can comprise a cell surface marker or a secreted factor. In any of the embodiments disclosed herein, the immune marker can be labeled with a detectable marker comprising a fluorescent marker, a bioluminescent marker, a colorimetric marker, and a radioactive marker. In any of the embodiments disclosed herein, the immune marker can comprise a cell surface marker or a secreted factor.

In any of the embodiments disclosed herein, the fluid chamber further can comprise a fluid medium suitable for growth and/or expansion of the immune cell.

In any of the embodiments disclosed herein, the black box engineering model used to predict the change in inflammatory state of the immune cell can be include or be constructed from a finite impulse response (FIR) model, an autroregressive with exogenous input terms (ARX) model, an autoregressive-moving-average (ARMA) model. The black box model may be constructed from an orthogonal basis function, such as a Laguerre series basis function. These functions may be combined in either linear or non-linear configurations.

### EXAMPLES

The present disclosure is also described and demonstrated by way of the following examples. However, the use of these and other examples anywhere in the specification is illustrative only and in no way limits the scope and meaning of the disclosure or of any exemplified term. Likewise, the disclosure is not limited to any particular preferred embodiments described here. Indeed, many modifications and variations of the disclosure may be apparent to those skilled in the art upon reading this specification, and such variations can be made without departing from the disclosure in spirit or in scope. The disclosure is therefore to be limited only by the terms of the appended claims along with the full scope of equivalents to which those claims are entitled.

### Example 1: Development of a Dynamic Predictive Control Loop for Macrophages

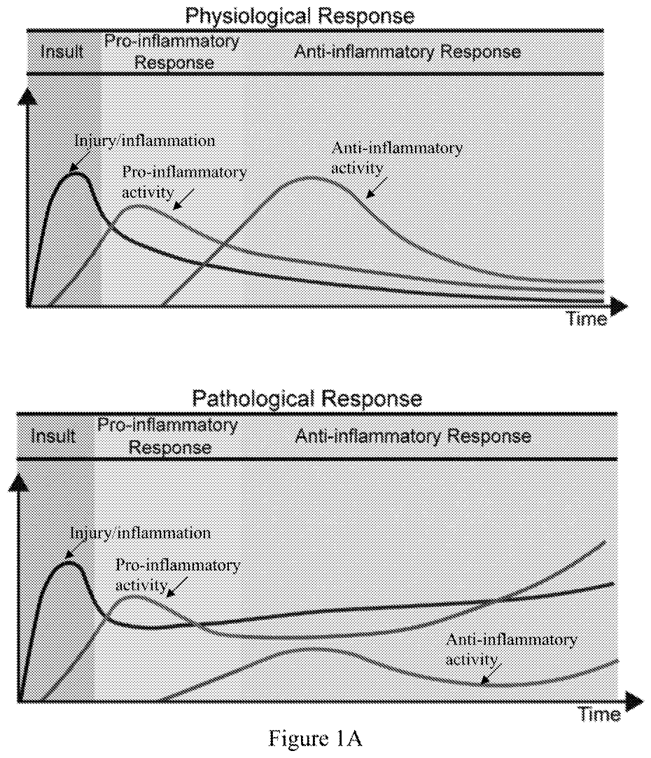

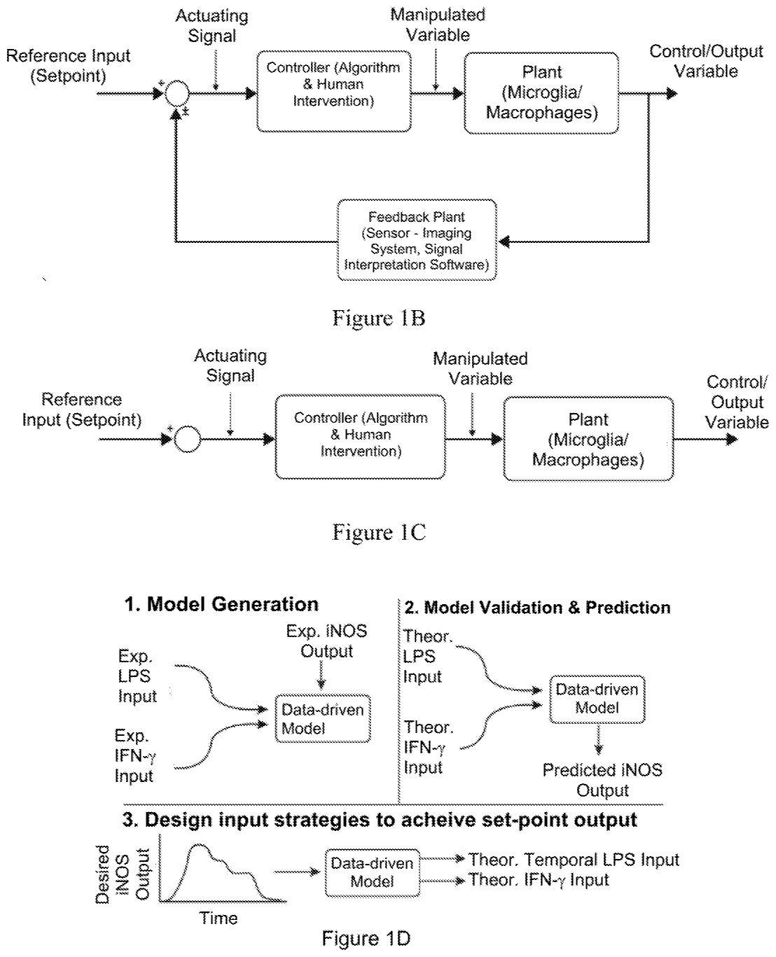

The inventors have formulated a data-driven modeling approach, informed by an in vitro macrophage polarization assay and system identification theory, to identify the temporal dynamics of macrophage response to multiple exogenous pro-inflammatory stimuli. Specifically, the inventors conditioned RAW 264.7 macrophages with M1 polarizing stimuli (e.g., LPS and IFN-γ) and quantified response in terms of iNOS expression for 1-72 hr post-stimulation. We then used least squares regression to fit a low-order autoregressive with exogenous terms (ARX) model together with nonlinear elements to relate iNOS response to each input (FIG. 1D, panels 1-2). The model identified predicted the dynamics of polarization in subsequent experiments in response to different concentrations and temporal trajectories (simultaneous vs sequential) of each input (FIG. 1D, panel 3). Finally, the inventors used the identified model as part of an open-loop control framework to tailor input sequences to achieve desired temporal trajectories of macrophage polarization in vitro. This study demonstrates that it is possible to experimentally control immune cell dynamics using a predictive control framework. Given the importance of dynamic M1 and M2 polarization during tissue regeneration, the control methodology presented here defines a novel framework that will have diverse applications for treating chronic inflammatory diseases and promoting tissue regeneration.

### Methods

Raw 264.7 Macrophage Cell Culture and Conditioning.

All studies in this work were performed using RAW 264.7 murine immortalized macrophages (ATCC TIB-71™). Macrophages were expanded, maintained, and cultured in basal macrophage medium, which is comprised of DMEM (Thermo Fisher Scientific; Ser. No. 12/430,062), 10% FBS (Thermo Fisher Scientific; 26140079), and 1% antibiotic/antimycotic (Sigma-Aldrich; A5955).

Raw 264.7 macrophages were cultured to 70% confluence before conditioning began. Cells were conditioned by addition of medium with lipopolysaccharide (LPS; Sigma-Aldrich; L2880), interferon gamma (IFN-γ; R&D Systems; 485-MI), or interleukin (IL)-4 (PeproTech; 214-14) as indicated.

Raw 264.7 macrophages were conditioned with LPS or IFN-γ alone to quantify individual stimulus dynamic response, with LPS or IFN-γ sequentially to recover iNOS expression via orthogonal input, or with LPS or IFN-γ simultaneously to quantify supra-additivity and model predictive control strategy response. Pre-treatment, 24 hours of IL-4 prior to addition of LPS or IFN-γ, was used to induce an anti-inflammatory, non-nave state for experiments involving hysteretic effects.

Quantification of iNOS Expression Via Immunofluorescence and Western Blot.

For immunocytochemistry (ICC) experiments, macrophages were cultured in 96-well microplates. Macrophages were fixed in 4% PFA solution for 15 minutes and blocked with 5% BSA+3% goat serum in PBS for one hour. Cells were stained with α-iNOS antibody (Cell Signaling Technology; Cat. No. 13120; 1:400) and DAPI for normalization to nuclei count. Cells were imaged at 10× magnification (Zeiss Observer Z1). Image fluorescence was thresholded and total fluorescence above the threshold was normalized to nuclei number.

For Western blot experiments, cells were cultured in 6-well plates then lysed using RIPA buffer with PMSF (Sigma-Aldrich), and cOmplete Mini (Sigma-Aldrich). Membranes were probed for α-tubulin (Sigma-Aldrich, Cat. No. T6074; 1:4000) and iNOS (1:1000). Membranes were imaged on a LiCor Odyssey CLx machine and quantified in ImageStudio Lite. iNOS band intensity was normalized to α-tubulin intensity to yield iNOS expression.

Data Normalization and Dynamic iNOS Response Figure Generation.

ICC and Western blot data were aggregated and iNOS expression for each independent experiment was normalized to the positive control with Raw264.7 cells treated with 1 μg/mL LPS for 24 hours (dynoDataLoad.m). iNOS dynamics plots were generated using the Gramm package for MATlAB. Data at sampled time points (0, 24 48, and 72 hours) were expressed as mean±SEM for separated data (N=38 for LPS single input experiments; N=8 for LPS repeated input experiments; N=8 for LPS cycled input experiments; N=32 for IFN-γ single input experiments; N=16 for IFN-γ repeated input experiments; N=16 for IFN-γ cycled input experiments). To generate interpolation curves data were smoothed using the Savitzky-Golay (sgolay) option in the curve fitting toolbox. Shaded band on curve represents root mean squared (RMS) cross validation error on smoothed data (macrophageDyn_figGenv3.m).

SISO and MISO Linear ARX Model System Identification.

LPS response data were compiled into a time-domain data object with experiments for all input concentrations and unique input sequences. Dynamic models were fit (Table 1) to the autoregressive with exogenous inputs (ARX) model structure

A(z)y(t)=B(z)u(t)+ε(t) (1)

where u(t) is the LPS stimulation input, y(t) is the iNOS response, and the model coefficients consist of

A(z⁻¹,θ)=1+a₁z⁻¹+a₂z⁻²+ . . . +aₙz⁻ⁿ^{ₐ} (2)

B(z⁻¹,θ)=b₀+b₁z⁻¹+b₂z⁻²+ . . . +bₙ_{_{b}}z⁻ⁿ^{_{b}} (3)

with one poles (nₐ), two zeros (n_{b}), an input-output delay of 1 time step, 24 hour time step, and zero initial conditions (System Identification toolbox, MATLAB). Parameters were estimated by solving the least squares problem

(J^{T}J)θ=J^{T}y (4)

where J is the regressor matrix with given inputs and y is the measured output, and the uniquely identified solution to the least squares parameter estimation is

θ=[a₁a₂ . . . aₙ_{ₐ}b₀b₁ . . . bₙ_{_{b}}]^{T} (5)

The sampling time step of identified model was set to 24 hours, which was equal to the data acquisition time step.

Realized for control design and flow diagram integration, the canonical controllable state space equations for this ARX model are of the form Eqs. 6 and 7 with matrix coefficients listed in Table 2.

x(t+1)=Ax(t)+Bu(t) (6)

y(t)=Cx(t)+Du(t) (7)

Where A is the system matrix, B is the input matrix, C is the output matrix, D is the feedthrough matrix, and t is time. Model order was selected to minimize the small sample-size corrected Aikike's Information Criterion (AICc) and mean squared error (Table 3, Table 4). This process was repeated for a SISO IFN-γ model (nₐ=1, n_{b}=2, and nₖ=1) and a multi-input single output (MISO) model with both LPS and IFN-γ inputs (nₐ=1, n_{b}=2 for both inputs).

TABLE 1
LPS ARX polynomials.

| | | z⁰ | z⁻¹ | z⁻² |
|---|---|---|---|---|
| LPS | A | 1 | −0.3163 | — |
| | B | 0 | 0.81 | −0.7727 |
| IFN-γ | A | 1 | −0.3849 | — |
| | B | 0 | 0.0634 | 0.0566 |
| LPS + IFN-γ | A | 1 | −0.76 | — |
| | B1 | 0 | 0 | 1.252 |
| | B2 | 0 | 2.019 | 0 |

TABLE 2
LPS transfer function.

| | | LPS Model | IFN-γ Model | LPS + IFN-γ Model |
|---|---|---|---|---|
| A | A₁₁ | 0.3163 | 0.3849 | 0 |
| | A₁₂ | 0 | 0 | 0 |
| | A₂₁ | 0.5 | 0.5 | 1 |
| | A₂₂ | 0 | 0 | 0.76 |
| B | B₁₁ | 2 | 0.5 | 0.6262 |
| | B₁₂ | — | — | 0 |
| | B₂₁ | 0 | 0 | 0 |
| | B₂₂ | — | — | 1.009 |
| C | C₁ | 0.405 | 0.1268 | 0 |
| | C₂ | −0.7727 | 0.2263 | 2 |
| D | D₁ | 0 | 0 | 0 |
| | D₂ | — | — | 0 |

TABLE 3
LPS ARX model AICc
for parameter number, nₐ and n_{b}, ranging from 1-4.

| | | nₐ | | | |
|---|---|---|---|---|---|
| | AICc | 1 | 2 | 3 | 4 |
| n_{b} | 1 | 331.62 | 430.59 | 548.77 | 707.25 |
| | 2 | 425.95 | 383.23 | 561.34 | 697.86 |
| | 3 | 550.49 | 562.75 | 574.56 | 711.95 |
| | 4 | 640.84 | 683.44 | 697.82 | 1789.39 |

TABLE 4
LPS ARX model MSE parameter number, nₐ and n_{b} ranging from 1-4.

| | | nₐ | | | |
|---|---|---|---|---|---|
| | MSE | 1 | 2 | 3 | 4 |
| n_{b} | 1 | 0.10 | 0.01 | 0.12 | 0.22 |
| | 2 | 0.04 | 0.01 | 0.12 | 0.21 |
| | 3 | 0.14 | 0.13 | 0.13 | 0.21 |
| | 4 | 0.17 | 0.20 | 0.20 | 0.21 |

LPS System Controller Design.

Controller design was carried out in the Control System Designer application (MATLAB, Mathworks) to find an input strategy capable of achieving the unit step response from a step reference. Since the estimated system dynamics indicated a continuous time zero at the origin, the inventors selected a PI controller to compensate because it adds a continuous time pole and is widely used in engineered systems. A proportional-integral (PI) controller (time domain equation (Eq. 8) and transfer function form (Eq. 9), was designed with robust noise and quick response specifications (parameters given in Table 5).

u(t)=Kₚe(t)+KᵢΣ₀^{t}e(t) (8)

$${u}_{c} = {K}_{p} + \frac{{K}_{t} * {T}_{s}}{2} \frac{z + 1}{z - 1} \qquad ( 9 )$$

Additionally, since the system model, Eq. 1, enabled state estimation, the inventors implemented a third order linear-quadratic Gaussian (LQG) controller, defined to minimize J

\tilde{J}=Σₜ₌₀^{N-1}(xₜ^{T}Qxₜ+xₜ^{T}Ruₜ)+x_{N}^{T}Q_{f}x_{N} (10)

The controller was tuned to be robust to noise and assuming moderate measurement noise (zero/pole/gain parameters in Table 6). where N is the time horizon, t is the time step, Q is the state cost matrix, Q_{f} is the final state cost matrix, and R is the input cost matrix. Q, Q_{f}, and R were defined internally by the system designer application.

TABLE 5
PI controller parameters

| | Value |
|---|---|
| Kₚ | 0.401 |
| Kᵢ | 0.0334 |
| Tₛ | 24 |

TABLE 6
LQG controller design

| | Value |
|---|---|
| Z | −2.631; 0.4089 |
| P | 1.0; 0.9539 |
| K | 0.1966 |

Surface Interpolation for Nonlinear Model Elements Parameterization. Supra-Additive Pro-Inflammatory Surface.

Data matrices across concentration gradients of simultaneous LPS and IFN-γ addition were divided by the iNOS expression level given LPS only for each concentration to give the ratio by which each IFN-γ concentration amplifies iNOS expression. The discrete matrix data were fit using cubic interpolation (Curve Fitting Toolbox) for each sampled time point. The resulting scaling factor, 0.1, can be queried for intermediary concentrations of each input at each sampled time.

M2 Hysteresis Surface.

iNOS expression for non-M2 polarized LPS-only treated cells were divided, for each respective LPS concentration, by expression by cells treated with an array of IL-4 concentrations for 24 hours followed by 24 hours of LPS. The matrix of LPS and IL-4 concentrations was interpolated using 3^{rd} order linear least squares, which provided inverse of the continuous input concentration-dependent attenuation factor γ. The y factor is inverted before being returned.

Global System Model Architecture and Formulation.

For the first nested model, the inventors used a multiple regression with interaction terms to quantify the supra-additive effect of adding both IFN-γ and LPS. Simulations were run using SISO models for single- and double-stimuli experimental results to populate a table with predicted output levels for varying magnitudes of input. The linear dual-input (both IFN-γ and LPS for all time points) model predictions were used as the regression output y, and the single input (either IFN-γ or LPS) SISO model predictions were given as regression inputs to fit a model of relative contributions of time and input interactions (y_{LPS}′ and y_{IFNγ}′). The terms that significantly predicted total iNOS output y were time-dependent LPS concentration, time-dependent IFN-γ concentration (Eq. 11). Weighting coefficients, c, for each term are given in Table 7.

y=c₁ty_{LPS}′+c₂ty_{IFNγ}′+c₃y_{LPS}′y_{IFNγ}′ (11)

TABLE 7
Multiple regression 1 interaction terms, coefficients, and p-values.

| Term | Estimate | p-value |
|---|---|---|
| Time:LPS-induced iNOS | 0.1696 | 1.299e−08 |
| Time:IFN-γ-induced iNOS | 0.3458 | 6.485e−07 |
| LPS-induced iNOS:IFN-γ-induced iNOS | 69.738 | 7.366e−11 |

The inventors next sought to construct a second global model structure that handles time- and concentration-dependent supra-additive interaction terms. Here, experimentally obtained data of iNOS expression given varying concentrations of LPS and IFN-γ was fit to a response surface, as described above, for each time point. This surface was used to define a table as above but with improved time and input-dependent dual-input model output predictions. A multiple linear regression on this prediction table similarly fit coefficients for time and input interaction terms (Eq. 11, Table 7). The inventors accounted for this temporally shifting interaction term by implementing the multiple linear regression model with the output from the identified SISO transfer function models and time as inputs and the MISO transfer function output as multiple regression model output.

Global System Model MPC Controller Design and Prediction.

The Model Predictive Control toolbox in MATLAB was used to create the controller and define manipulated input sequences for the MISO “global” model. The SISO IFN-γ and LPS transfer functions with weighting coefficients derived from the multiple regression was given as the model object, referred to as the plant (Eq. 12, FIG. 1D). The plant model was defined with two manipulated variable inputs, one output, a control horizon of 72 hours, and a prediction horizon of 120 hours. Manipulated variables were constrained with a minimum of 0, a maximum of 1, and unconstrained rates of change. The default state estimator (Kalman filter) settings were used for the controller predictions (MATLAB). Closed loop simulations generated the inputs, u, needed to obtain the set reference (unit step) over simulation time with the expected system output y. Plant performance was evaluated by running open-loop simulations given the predicted inputs from the closed-loop simulation. Optimal predicted input and output trajectories were validated using the mpcmove function.

G=(C₁Y₁+C₂Y₂)+C₃Y₁Y₂ (12)

### Results

Macrophage iNOS Expression is Transient and Refractory to Repeated Stimulations.

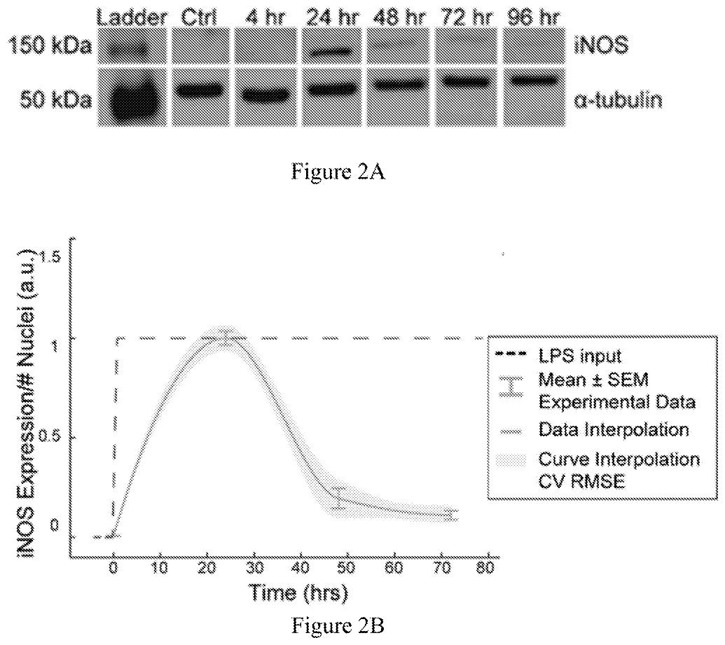

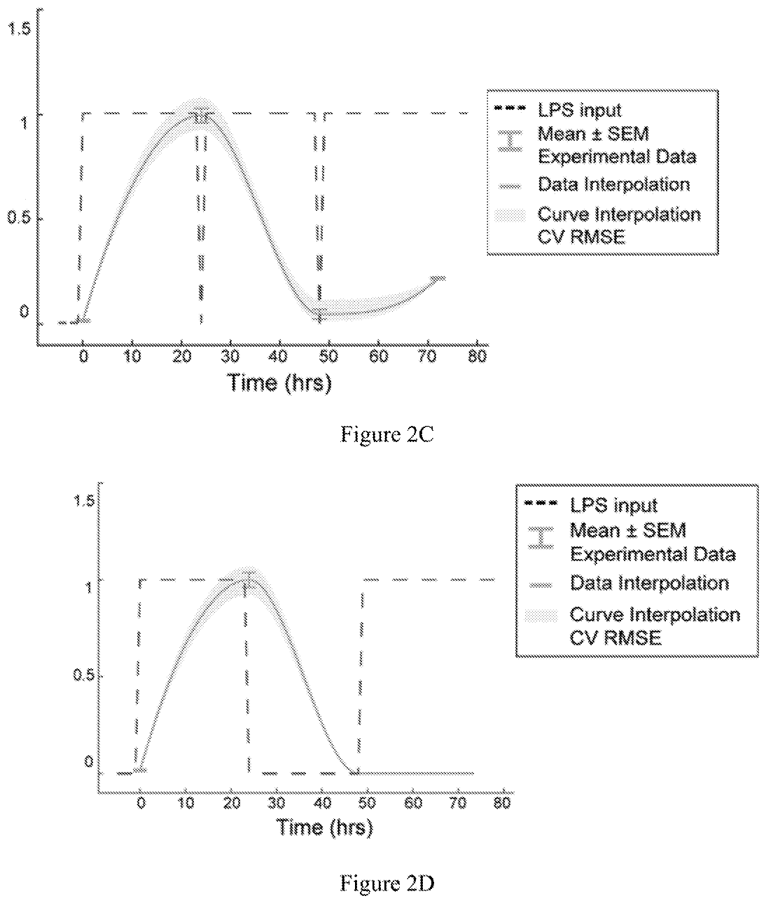

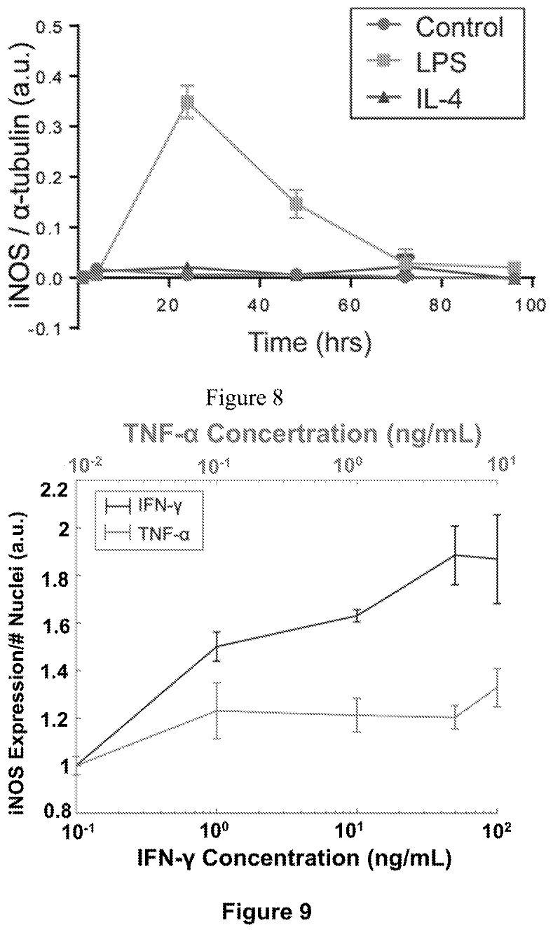

The inventors first aimed to determine the temporal dynamics of macrophage response to single or repeated pro-inflammatory stimuli. As a model system, the inventors used expression of the pro-inflammatory M1 marker inducible nitric oxide synthase (iNOS) by Raw 264.7 macrophages in response to the pro-inflammatory stimulus lipopolysaccharide (LPS). Using quantitative Western blot, it was found that a single administration of 1 μg/ml LPS, but not IL-4, resulted in transient iNOS dynamics with a peak in iNOS expression at 24 hr followed by a decay to baseline over the following 48 hr (FIG. 2A). Immunocytochemistry (ICC) confirmed this response (FIG. 2C) and revealed that this temporal trajectory was 1) conserved given a range of lower doses of LPS and 2) that the magnitude of the response monotonically increased with the magnitude of the stimulation (FIG. 8). Intriguingly, although LPS was not removed from cultures, and thus represented a persistent step-like stimulus, the dynamics of iNOS expression followed a first order decay response (FIG. 2C). In traditional engineered systems, this type of system response is stimulating cells with LPS following 24 hours in control media. However, cycled re-stimulation did not alter iNOS expression dynamics (FIG. 2D), suggesting that the dynamics of macrophage polarization to LPS stimulation consist of an initial response that is not sustained despite either continued or repeated LPS stimulation, i.e., the system becomes refractory. This refractory behavior resembles tolerance/fatigue observed in chronic disease conditions, such as type 2 diabetes and cancer.

Auto-Regressive Model with Exogenous Inputs Fits iNOS Dynamic Response to LPS Input.

To determine if it is possible to recover and sustain iNOS expression, and, by extension, pro-inflammatory activation of Raw264.7 cells, the next used a control systems engineering methodology to design a temporal sequence of LPS stimulation. Control systems methodology often requires a model that can be used to predict future system response given a known stimulation input.

Diverse model structures are employed in engineering fields, ranging from high-ordered mechanistic models to input-output data-driven models. For this application, a mechanistic model encoding all of the genetic and protein interactions responsible for iNOS expression might suffer from reduced predictive capacity due to uncertainty in fitted parameters. Grey and black box models, which capture dominant response dynamics without specifying mechanistic details, may be more appealing to relate iNOS dynamics to pro-inflammatory stimulation. The inventors therefore sought to identify an optimized black box single input and single output (SISO) model relating LPS input to iNOS output. A critical tradeoff should be considered when choosing model structure: maximizing flexibility to best capture system dynamics while lessening the need to have more model parameters than can be reliably identified from the data. Autoregressive models with exogenous inputs (ARX) models are frequently used for black-box system identification because they can capture underlying system dynamics in diverse applications and parameterization using the ARX structure guarantees uniqueness of solution and identification of the global minimum of the error function.

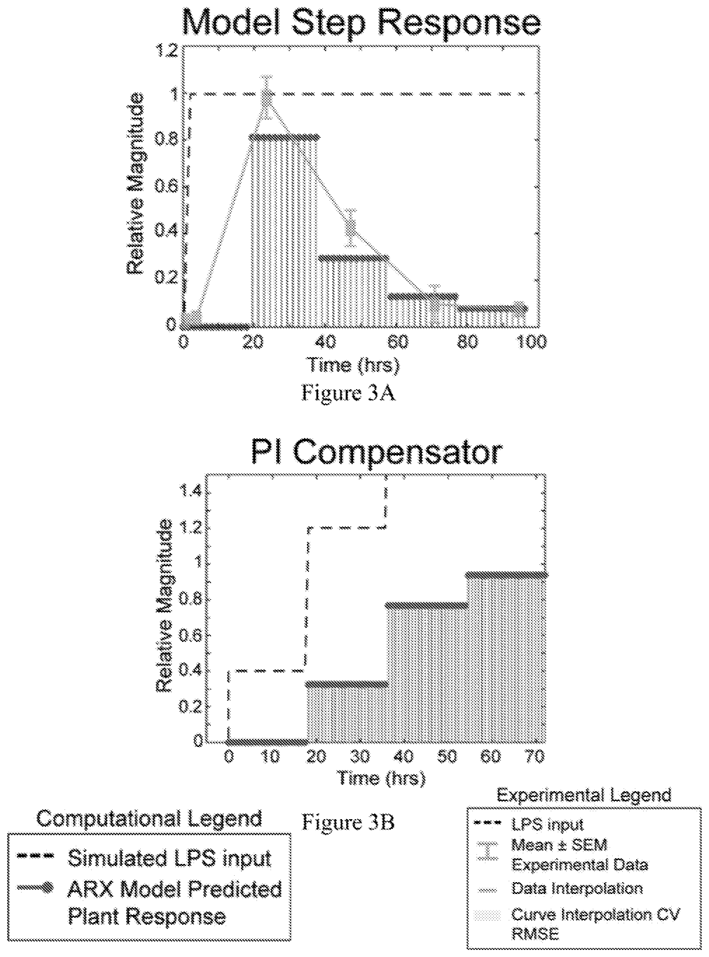

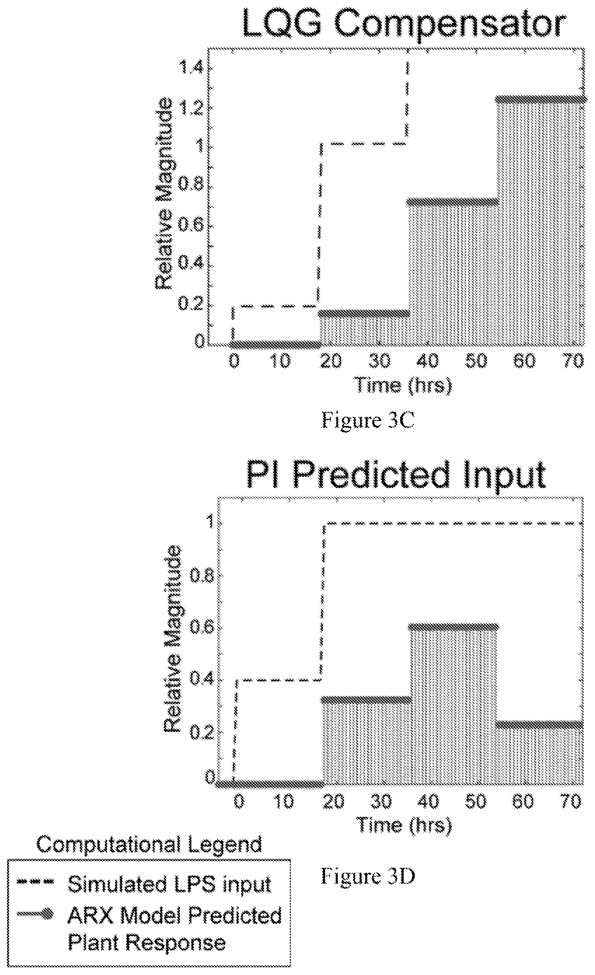

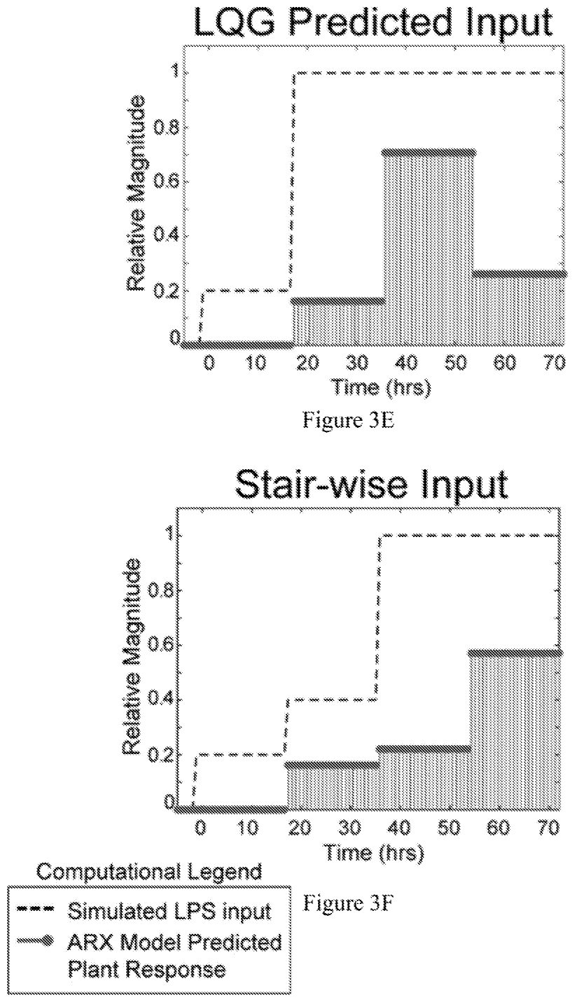

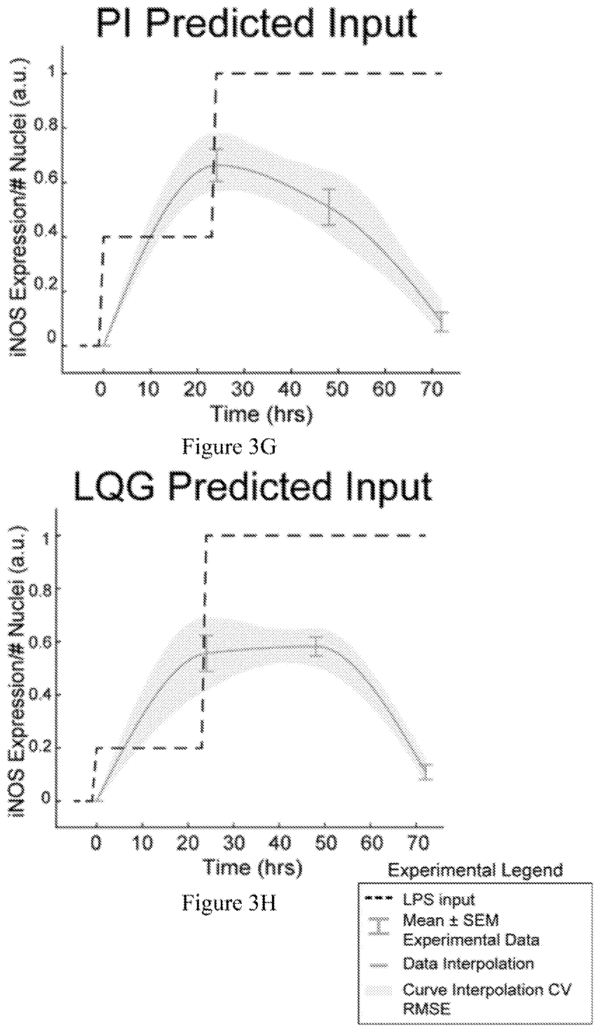

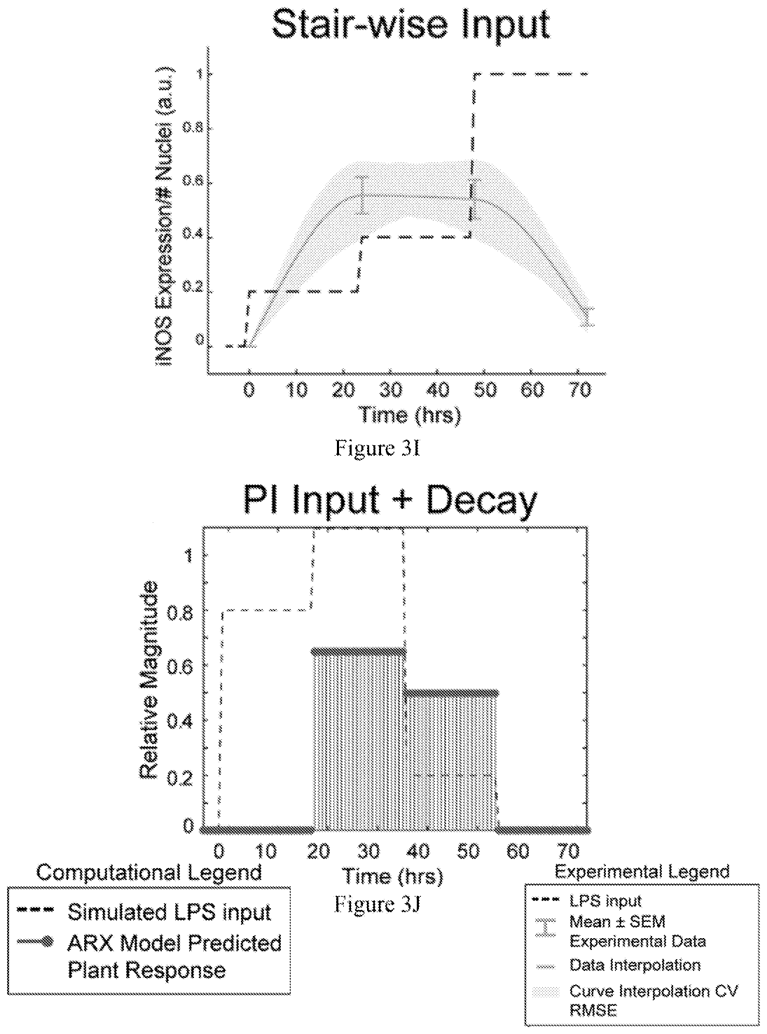

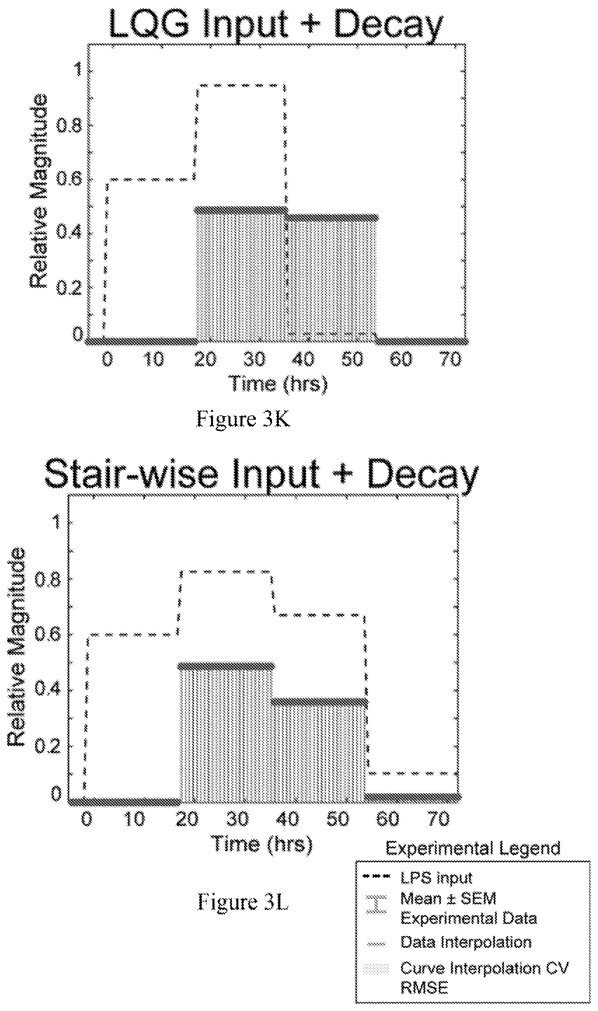

To identify the parameters of this model architecture, extensive characterization of macrophage polarization dynamics with multiple input patterns and magnitudes was performed to generate a rich data set to train and identify an input/output model of iNOS expression dynamics (FIGS. 2B-2D; FIG. 8). It was experimentally found that macrophages exhibited a monotonic LPS dose-to-iNOS response relationship within a physiologically relevant concentration range (FIG. 8), which is well-described using the linear ARX model structure. Above a high (1 μg/ml) concentration of LPS, response tapered off, potentially due to cell death or changes in intracellular signaling activity. As such, the inventors set 1 μg/ml LPS as the maximum concentration used in this study. To capture the post-LPS stimulation refractory period, the inventors selected an ARX model order (na=1, nb=2, nk=1) that recapitulated this refractory pattern for a step input (FIG. 3A). The model parameter estimates are given in Table 1 (three free coefficients) and returned a normalized Aikike's Information Criterion (AICc) model quality metric of 430.59. This model outperformed the related ARMAX (autoregressive-moving average with exogenous terms) model structure with similar numbers of parameters (nₐ=1, n_{b}=2, nₖ=1; AICc=501.96). By estimating this input/output model, the inventors can achieve both high descriptive and predictive capacities.

Model Predictive Controller Identifies LPS Stimulation Sequence to Sustain iNOS Expression.

Using the identified ARX system model, the inventors sought to tune a controller (Control System Design Toolbox, MATLAB), placed upstream of the plant (FIG. 1C), that would predict a temporally defined LPS input strategy to overcome the persistent decay in iNOS expression. The inventors used two controller structures to design input strategies capable of achieving sustained iNOS expression. First, since the system dynamics (FIG. 2B) indicated that the system is responding to the derivative of the input, the inventors attempted to compensate for the derivative using a classical proportional-integral (PI) controller, which is commonly applied in engineering application to minimize steady-state error (Table 5). Here, the inventors used the PI controller to control LPS-induced iNOS expression to the unit reference (1 a.u. iNOS relative expression). The controller predicted that a stair-wise delivery of LPS (FIG. 3B, dashed line) would give rise to a more gradual but prolonged output y response that reached the reference by the control horizon of 72 hours (FIG. 3B, gray stems). Importantly, the second step in input exceeded the unit input value (corresponding in vitro to LPS), which was the upper bound of LPS concentration used in this study. When the controller was constrained to inputs between 0 and 1 (1 μg/ml LPS) no PI controller obtained by adjusting Kₚ and was capable of defining an input sequence that both maintained a u≤1 μg/ml and predicted y to reach the reference the control time horizon.

Due to the inability of the PI controller to identify an input sequence capable of reaching or maintaining output levels at 72 hours, the inventors next decided to take advantage of the ARX system model to re-designed the input sequence using a third order linear-quadratic Gaussian (LQG) controller (Table 6), which can provide improved performance over conventional PID controllers for minimizing total error. This LQG controller designed a reduced magnitude for the original input followed by the unit max of LPS input (FIG. 3C, dashed line) to achieve the 80% of reference point (FIG. 3C, gray stems) that the PI controller defined input could not achieve within LPS concentration constraints. However, this controller also required u≥1 μg/ml to reach the reference. When the input is constrained to 0≤u≤1 μg/ml LPS, the model simulations predicted that progressive step increases in LPS would prolong the iNOS response but not sustain it at the unit reference value (FIGS. 3D-3E). When the initial magnitudes of the LQG and PI predicted inputs are heuristically combined in a three-step increase strategy, simulations do predict a maximum response at 72 hours (FIG. 3F).

The controllers designed for each model architecture defined a temporally increasing magnitude of u, or LPS concentration, where the input is increased at each time step. Experimentally, the model predicted input values represent a fraction of the normalized maximum (high) LPS concentration, 1 μg/ml. For example, 0.2 is 20% max or 20 ng/ml, and 0.4 is 40 ng/ml. To test the PI controller input strategy, Raw 264.7 macrophages were treated with 40 ng/ml of LPS for 24 hours, followed by 1 μg/ml from hour 24 until fixation at 72 hours (FIG. 3G, dashed line). Despite the computational prediction of a u of 1.2, biologically this would have led to excessive cell death, likely changing the plant response. Thus, the inventors tested the effect of the unit max of LPS in this stair wise input scheme. The macrophage expression of iNOS peaked at approximately 70% of normalized max iNOS (defined by the 24 hour expression level given 1 μg/ml LPS) at 24 hours (FIG. 3G, gray curve). The subsequent increase in LPS concentration delivered did not sustain this level of iNOS, which declines through the 48 and 72 hour time points, but did keep levels higher (˜50% max) at 48 hours than an initially high level of LPS (FIG. 3G, gray curve).

The LQG controller predicted input, 24 hours of 20 ng/ml followed by 48 hours at 1 μg/ml LPS (FIG. 3H, dashed line), realized an iNOS expression level 60% of the reference at 24 hours (FIG. 3H, gray curve). Intriguingly, here the cells sustained this iNOS level through 48 hours, but not through 72 hours (FIG. 3H, gray curve). The inventors next heuristically combined the input strategies defined by the PI and LQG controller to test whether iNOS expression at 72 hours could be sustained (FIG. 3I, dashed line). However, iNOS expression given this strategy reflected that of the LQG controller and did not keep activation high at 72 hours FIG. 3I, gray curve).

The refractory or muted iNOS response to either high, continued or step-wise increases in LPS stimulation suggested a decaying efficacy of LPS regardless of input sequence. Indeed, when the input sequence terms were multiplied by a time-dependent exponential decay vector (FIGS. 3J-L, dashed lines), the response magnitudes reflect the experimentally obtained iNOS values (FIGS. 3J-L, gray stems) for each input strategy. Though this single input system was unable to meet control specifications, the ability to qualitatively maintain elevated pro-inflammatory macrophage activation via the inventors' predictive control framework demonstrated feasibility of the approach, possibly extendable to more advanced systems that can overcome the decaying efficacy of LPS stimulation.

IFN-γ Stimulation Increases Reachable iNOS Trajectories and Adds System Nonlinearity.

Single or repeated stimulation with LPS was unable to sustain iNOS expression and sustained expression was only partially recovered by temporally modulating the input (FIGS. 3D-3I), i.e., inflammatory activity was modulated but could not be prolonged indefinitely. In engineering systems, independent inputs increase the system rank and thereby increase state achievability. That is to say, adding a secondary stimulus that operates through separate, orthogonal means, expands the internal states and reachable output of a system. Therefore, the inventors next hypothesized that a second pro-inflammatory input would improve controllability.

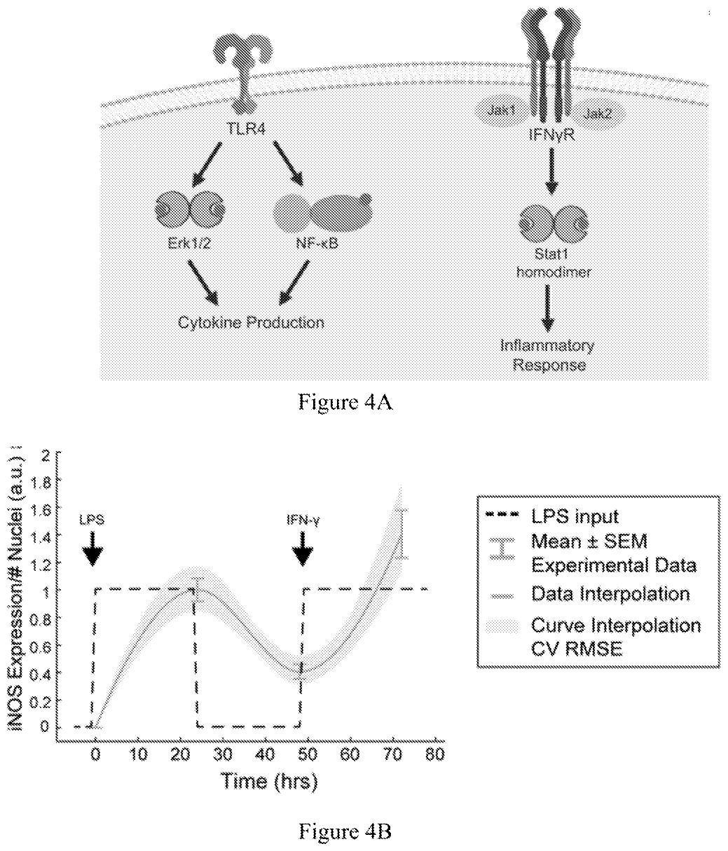

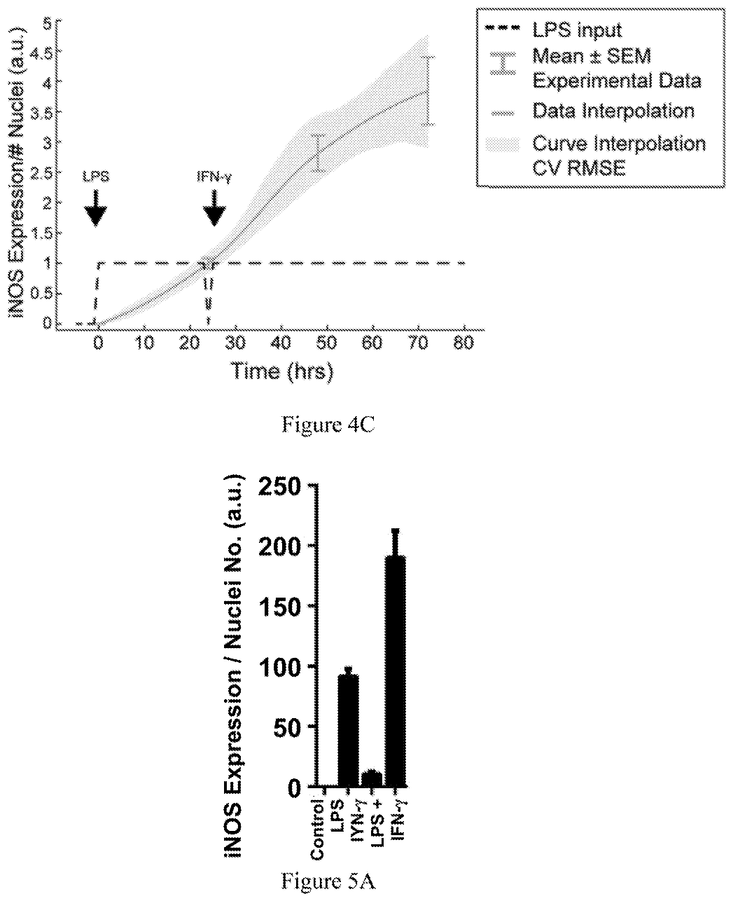

The inventors used IFN-γ, which signals largely independently of LPS (FIG. 4A) as the second, orthogonal input because IFN-γ robustly increased iNOS levels despite prior LPS input (FIGS. 4B-4C). Although TNF-α was also considered as the second pro-inflammatory stimulus, we found the iNOS response is more sensitive to IFN-γ within a physiologically relevant concentration range (FIG. 9). Given these findings, the use of multiple pro-inflammatory inputs is promising for toggling both the magnitude and duration of macrophage activity with greater reachability.

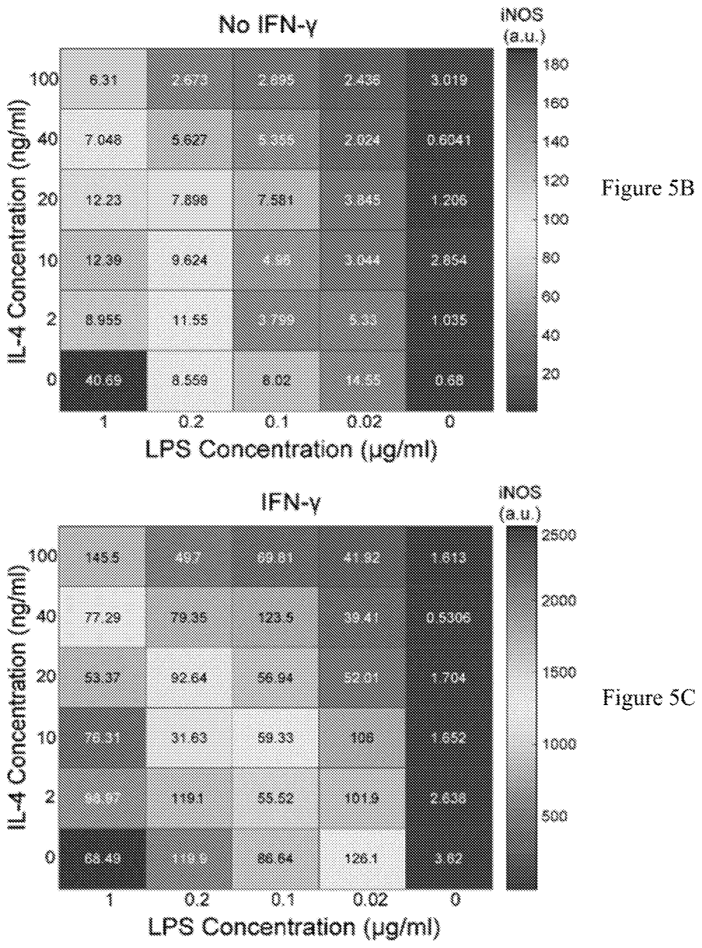

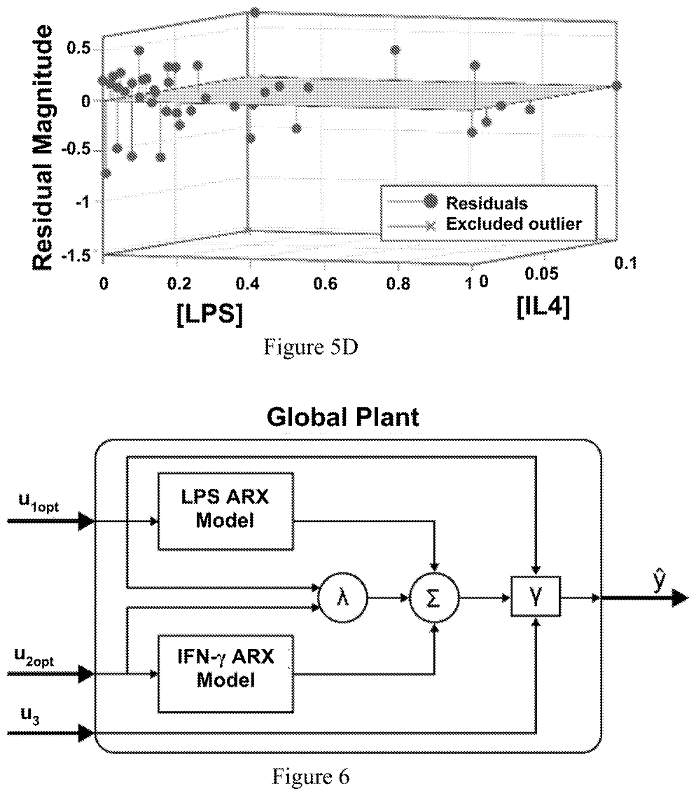

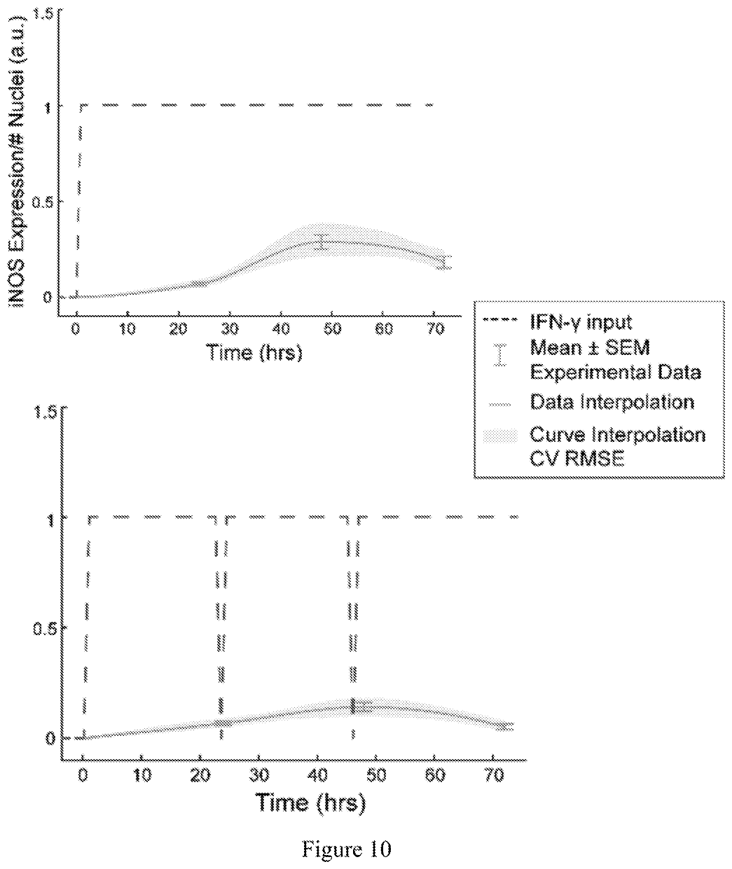

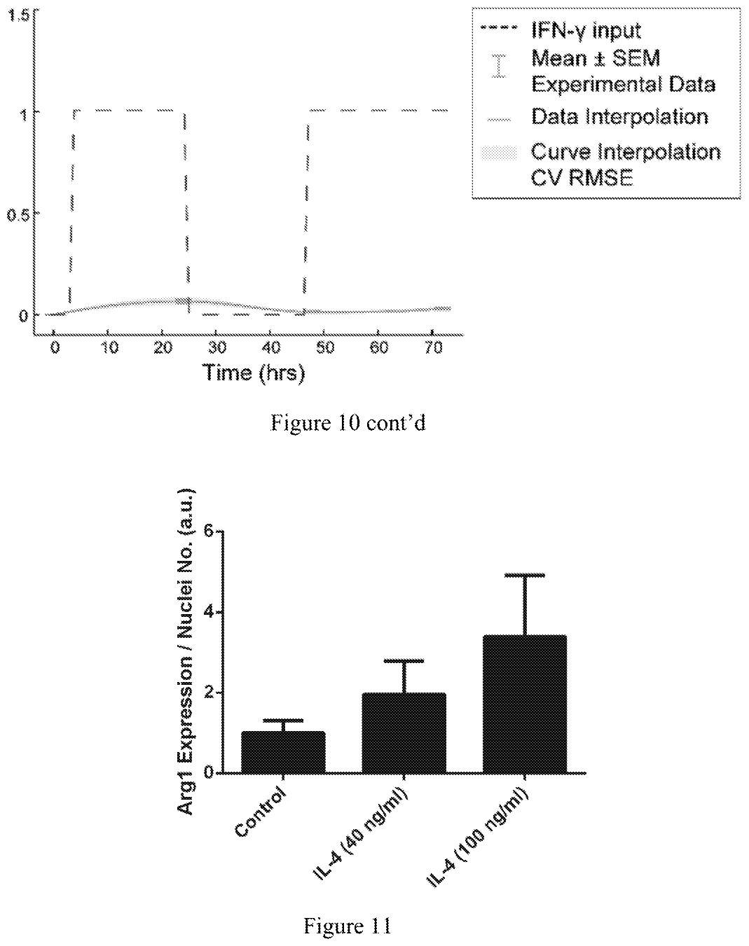

While IFN-γ recovered iNOS expression from LPS-induced tolerance, it also introduced a non-linear element to the dynamic response supra-additivity. ARX and transfer function models require that the output of the sum of two inputs equal the sum of the output of each input. However, IFN-γ amplifies LPS-induced iNOS expression, where expression is greater than the sum of expression from each stimulus alone, whether added concomitantly or in series. In fact, supra-additivity for simultaneous conditioning is present across all time points and for a range of LPS and IFN-γ concentrations through 72 hours in conditions (FIG. 5A, FIG. 10). The supra-additivity also leads to iNOS expression that is greater than the unit reference for 24 hours of LPS, so our predictive model will need to account for these nonlinearities to avoid overshooting or behavior that does not settled to the desired reference (FIG. 4C).

Raw 264.7 Macrophages Exhibit State Memory Based on Stimulation History.

In disease, macrophages may exist in chronically activated or other non-nave states, driven by local and systemic changes in signaling proteins, hormones, among other factors. Thus, having shown the ability to model macrophage pro-inflammatory dynamics and design input trajectories for nave macrophages, the inventors next wanted to determine whether the macrophage response to pro-inflammatory stimulation would be affected by pre-polarizing the cells toward an anti-inflammatory state.

To model Raw264.7 cells starting in a non-nave state, the inventors pre-conditioned macrophages with IL-4 for 24 hours prior to pro-inflammatory stimulation. Upon stimulation with LPS, it was found that prior IL-4 conditioning attenuated expression of iNOS after 24 hr of treatment with LPS, but that iNOS still responded to LPS in a concentration dependent mannerError! Reference source not found. For each LPS concentration, iNOS expression for non-M2 polarized LPS-only treated cells were divided by iNOS expression values from cell treated with an array of IL-4 concentrations for 24 hours followed by 24 hours of LPS. The matrix of LPS and IL-4 concentrations was interpolated using 3^{rd} order linear least squares, which provided the inverse of the continuous input concentration-dependent attenuation factor γ. M2 polarization was validated by increased expression of Arg1 (FIG. 11). Together, these data suggest that macrophages exhibit hysteresis in their response to prior inputs, whereby prior M2 polarization attenuates future M1 response and prior M1 polarization sensitizes future M2 response. The M2 driven attenuation of M1 response reflects the systemic immunosuppression that poses a major risk to post-traumatic or surgical injury patients.

Modeling Multi-Input Driven Hysteresis and Supra-Additivity.

Since the dynamics of iNOS expression in Raw264.7 cells were dependent on the polarization state history (i.e., hysteresis in non-nave cells) and demonstrated supra-additivity in response to combinations of LPS and IFN-γ, the inventors next sought to incorporate these elements into the iNOS response model. First, to account for prior IL-4-induced hysteresis, the inventors computed an attenuation factor, or the relative magnitude of iNOS expression for a range of LPS and IL-4 concentrations (100 ng/ml IL-4, 40 ng/ml IL-4, 20 ng/ml IL-4, 10 ng/ml IL-4, 2 ng/ml IL-4, 0 ng/ml IL-4) relative to expression with no exposure to IL-4. The attenuation factor, γ, is one for non-hysteretic systems and increases with higher concentrations of IL-4 such that 1/γ multiplied by iNOS expression for a given LPS concentration gives the iNOS response for that LPS concentration and an IL-4 pre-treatment concentration. A response plane for γ was fitted with a 3^{rd} order by 3^{rd} order polynomial to a smoothed continuous response surface from which any attenuation due to anti-inflammatory induction is returned (FIG. 5D).

To account for supra-additive effects of multiple pro-inflammatory inputs, as done for the hysteretic surface, the inventors populated time-dependent interaction term (2) surface curves for the defined ranges of co-addition of LPS and IFN-γ.

Excitingly, the supra-additivity of IFN-γ with LPS demonstrated the ability to recover the attenuation effect induced by IL-4. Indeed, greater iNOS expression was observed across lower LPS concentrations and higher IL-4 concentrations when IFN-γ is included compared against use of LPS alone (FIG. 5B-5C). This interaction effect demonstrated the need for a system plant model that processes both M2 and M1 inputs.

The global plant model was constructed and described schematically in FIG. 6. The system receives the concentration of LPS (u₁) and IFN-γ (u₂) which were passed into their respective identified ARX models, the supra-additivity of LPS and IFN-γ was accounted for using λ, the pro-inflammatory contributions were summed and applied as inputs to the hysteresis term y, Finally, the output was the predicted iNOS output (ŷ) as a function of time t (FIG. 6).

Design of LPS and IFN-γ Temporal Input Trajectories with Global Plant Model Achieves Sustained iNOS Expression.

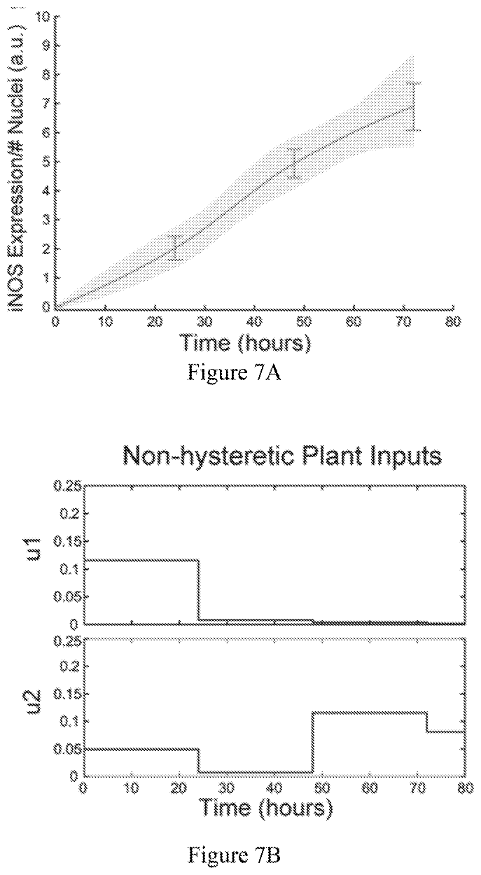

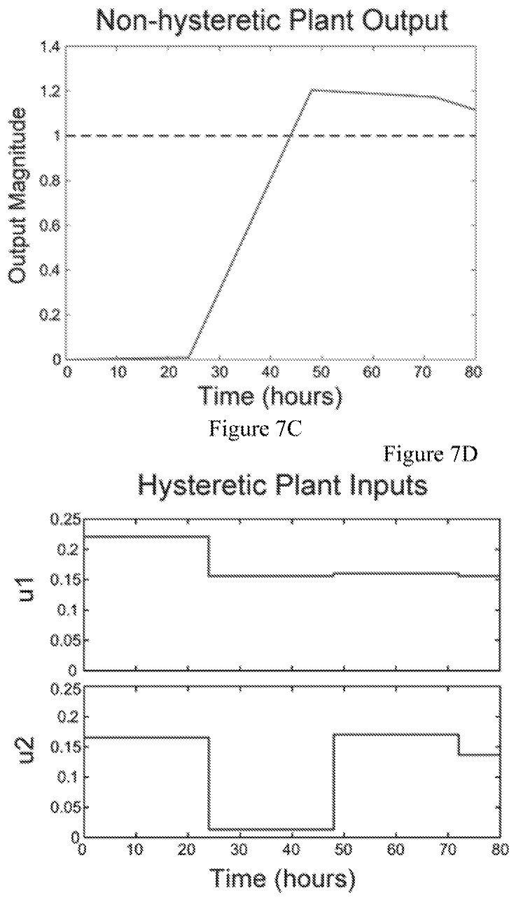

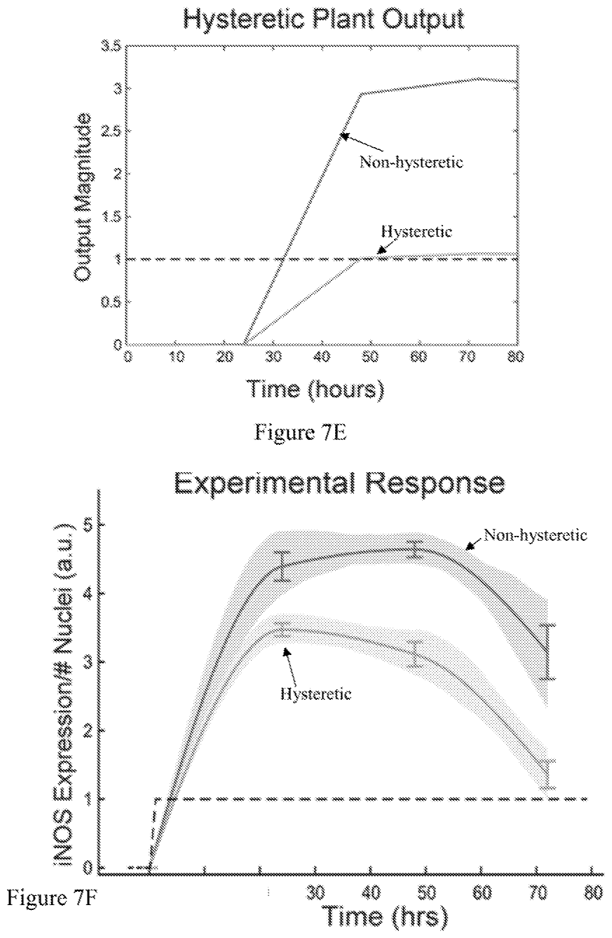

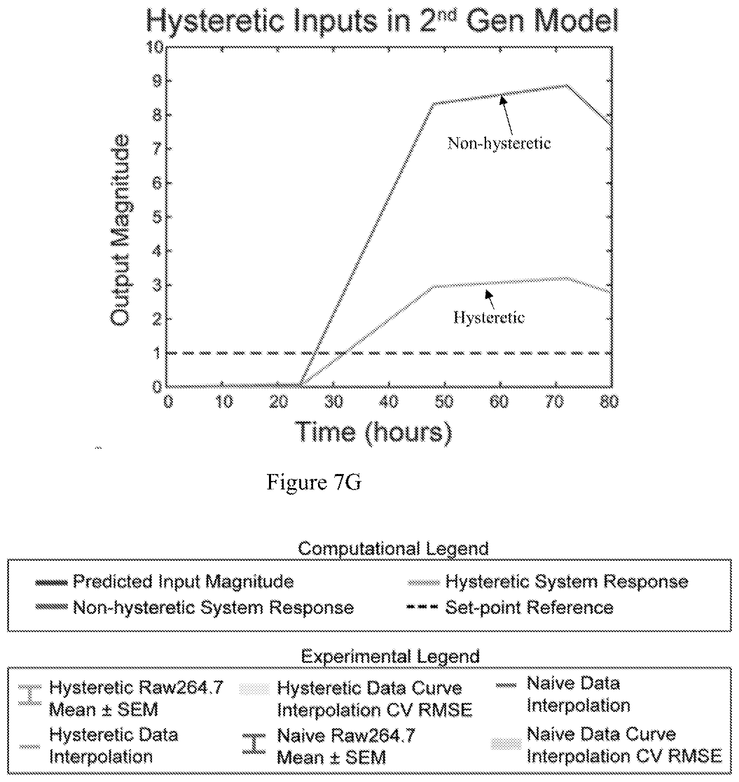

Transfer functions were linearly combined with regression coefficients for supra-additivity (λ) and hysteresis (γ) acting as pre-processing filters, i.e., the terms were multiplied with each model's output, then added. The global regression of the function has the final form in Eq. 11 (R²=0.748; p-value (vs. constant model)=1.34e-38). Simultaneous administration of unit, high, inputs in vitro vastly overshot the unit value of iNOS and did not settle over the course of the experiment (FIG. 7A). Using the global model, we used an MPC controller to design input trajectories LPS (u1) and IFN-γ (u2) needed to obtain sustained constant iNOS expression over a 72 hour control horizon (FIG. 7B). Using these trajectories the simulated plant reached the reference value by 24 hours with a minor overshoot and settled at approximately 96 hours (FIG. 7C). Including hysteresis in the plant controller estimation increases the predicted inputs magnitude for the unit step reference (FIG. 7D). Given the input sequence defined in (FIG. 7D), a hysteretic system is predicted to respond with relatively small overshoot and error (FIG. 7E, light gray bottom curve). Importantly, the model captured the large overshoot that would be expected from administering elevated input levels to a non-hysteretic system (FIG. 7E, dark gray top curve).

Next, the relative input magnitudes defined for a hysteretic plant (FIG. 7D) were translated to concentrations of LPS and IFN-γ, which were administered as temporally defined to Raw264.7 macrophages in culture. The macrophage iNOS expression trajectories reflected the model predicted response for both hysteretic, i.e. pretreatment with IL-4 (FIG. 7F, light gray bottom curve) and non-hysteretic (FIG. 7E, dark gray top curve) cell conditions. In total, these experimental findings show that our global plant model predicts the dynamics macrophage pro-inflammatory response, including transient response to LPS, supra-additivity, and hysteresis. Moreover, it was shown that this model could be used to define dual stimulation strategies that could prolong Raw 264.7 cell polarization as quantified by iNOS.

### Discussion

In this Example, the inventors have demonstrated a novel paradigm for engineering immune activity by defining predictive data-driven models of macrophage polarization and using them to define the dynamic delivery of pro-inflammatory factors to control the duration and magnitude of macrophage polarization. Rather than identifying detailed, highly parameterized mechanistic models, the inventors applied a control theory framework to globally describe the pro-inflammatory activity of macrophages over time. Specifically, using expression of canonical pro-inflammatory (M1) marker iNOS as an output, the inventors defined a black-box transfer function to capture the dynamic response of macrophages given a temporal sequence of applied LPS and IFN-γ as system inputs. The overall modeling framework coupled linear ARX models, which are uniquely identifiable, with nonlinear elements that accounted for state-history dependent hysteresis and supra-additivity from multiple pro-inflammatory stimuli. The inventors' global plant model structure not only predicted responses to different input sequences but enabled design of new stimulation sequences that yielded a desired temporal iNOS response without a refractory response (FIGS. 7A-7G).

Immune dysregulation plays a central role in diverse diseases. Dysregulated activity of macrophages in particular can both hinder tissue repair and promote disease pathogenesis. However, macrophage functional diversity and broad distribution throughout the body also makes them good targets for modulating immune function to treat an array of diseases. Yet the vast majority of new immunomodulatory strategies, including inflammatory agent inhibitors and cell-based therapies, do not explicitly account for the temporal evolution of macrophage response needed to resolve the response to injury.

The importance of a temporally dynamic immune response has been highlighted by recent findings that long term resolution of inflammation depends on a sufficiently pro-inflammatory initial response followed by an anti-inflammatory and resolving activity. Early pro-inflammatory macrophage response can enable clearance of pathogens and damaged cells and can subsequently trigger the anti-inflammatory and pro-regenerative response. Thus, the inventors sought to model and control macrophage pro-inflammatory activity, measured by iNOS expression. Using an ARX model structure, widely used for black-box system identification in engineering and biological systems, detailed above, the inventors identified computational models able to predict and control temporal iNOS expression. This black-box approach enabled the inventors to fit three parameters to model the dynamic LPS response and three more to fit the IFN-γ response, in contrast to dozens required in mechanistic differential equation models of macrophage polarization.

Interestingly, when implementing model-predicted LPS input sequences, the inventors observed that the time-dependent decay in the efficacy of LPS persisted. In fact, when the designed input magnitude was multiplied against a time-dependent decay term (FIGS. 3J, 3L, dashed lines), the inventors were able to simulate the observed experimental response. This finding is consistent with macrophage auto-regulatory processes that prevent runaway inflammatory activity to LPS.

The models can be further tuned for primary isolated macrophages. Further, to extend the utility of the model for disease therapeutics, similarities and differences between primary macrophages collected from wild type mice and mouse models of chronic inflammatory disease can be identified. For example, macrophages are known to exhibit distinct inflammatory profiles from diabetic patients than from healthy individuals, which can be reflected in the identified model parameters. Additionally, the methodology developed here lays a foundation for dynamic control of macrophage activation using a single polarization marker, but a wider panel of pro- and anti-inflammatory markers may be needed to fully delineate macrophage activation state and effector function.

The inventors' dynamic experimental and computational approach establishes a new way of conceptualizing and modulating macrophage activity by using a temporal sequence of input stimuli to shape the trajectory of inflammatory response. As shown herein, the inventors have experimentally validated the computational model predictions, extending previous theoretical work in model predictive control for patient-specific therapeutics. This framework may have broad-reaching applications both in vitro and in vivo. Moreover, the demonstrated ability to modulate macrophage activity suggests that design of temporally varying inputs has therapeutic potential for broad chronic inflammatory disorders.

The present disclosure is in no way limited to the hereinabove described embodiments. The present disclosure relates to one or more of the items as listed below, from 1 to 158:

1. A method for dynamic real-time modeling and/or control of an inflammatory response in an immune cell, comprising:

providing a fluid chamber comprising at least one inlet, at least one outlet, and the immune cell;

delivering a first stimulus through the inlet via a controller, the controller in fluid communication with the fluid chamber, wherein the stimulus elicits a change in the inflammatory state of the immune cell; and

detecting the change in the inflammatory state of the immune cell via a detector, the detector in fluid communication with the fluid chamber,

wherein the controller is configured to deliver a second stimulus based on the change in the inflammatory state of the immune cell in order to model and/or control the inflammatory response of the immune cell,

wherein the detector is configured to generate input and/or output data indicative of the change in the inflammatory state of the immune cell, and

wherein the change in the inflammatory state of the immune cell to each of the first stimulus and second stimulus is predicted by the steps of:
fitting a black box engineering model to the input and/or output data obtained by stimulating cells within the chamber; and
selecting a best fitting black box engineering model based on the input and/or output data and applying the best fitting black box engineering model to future input/output data.
   2. The method of item 1, wherein the fluid chamber is a cell culture chamber, a cell culture well, or a microfluidic chamber.
   3. The method of items 1 or 2, wherein the immune cell comprises at least one cell selected from the following types of cells: a microglial cell, an astrocyte, a macrophage, a B cell, a T cell, a natural killer (NK) cell, and a leukocyte.
   4. The method of items 1-3, wherein the immune cell comprises a microglial cell, a macrophage, or combinations thereof.
   5. The method of items 1-4, wherein the first stimulus comprises at least one immune-modulating molecule.
   6. The method of item 5, wherein the at least one immune-modulating molecule is pro-inflammatory or anti-inflammatory.
   7. The method of items 5-6, wherein the at least one immune-modulating molecule comprises an antigen, a cytokine, a growth factor, a sphingolipid, a complement factor, an immunomodulatory small molecule, an intracellular signaling inhibitor, an activator of pro-inflammatory or anti-inflammatory pathways, a cytokine inhibitor, and combinations thereof.
   8. The method of any of items 5-7, wherein a first immune-modulating molecule is administered at the same time as a second immune-modulating molecule.
   9. The method of any of items 5-8, wherein a first immune-modulating molecule is administered before a second immune-modulating molecule.
   10. The method of any of items 5-9, wherein the first immune-modulating molecule is administered between five minutes and 24 hours before the second immune-modulating molecule.
   11. The method of any of items 5-10, wherein a first immune-modulating molecule is different from a second immune-modulating molecule.
   12. The method of any of items 5-11, wherein a first immune-modulating molecule is the same as a second immune-modulating molecule.
   13. The method of any of items 1-12, wherein the first stimulus causes the immune cell to change from a pro-inflammatory state to an anti-inflammatory state.
   14. The method of any of items 1-13, wherein the first stimulus causes the immune cell to change from a quiescent state to a pro-inflammatory state.
   15. The method of any of items 1-14, wherein the change in the inflammatory state of the immune cell is detected by measuring a marker characteristic of the inflammatory state.
   16. The method of item 15, wherein a marker characteristic of the pro-inflammatory state comprises one or more of iNOS, SOCS3, TLR4, TLR2, IL-1R, MHCII, CD68, CD80, CD86, TLR2, TNFα, IL1α, ITAM1, IL1β, HIF1α, IL-12b, KCna3, GFAP, CLEC7a, Vimentin, CD69, CD27, CD45, CD44, and CCR7.
   17. The method of items 15 or 16, wherein a marker characteristic of the pro-inflammatory state comprises one or more of iNOS, SOCS3, TLR4, TLR2, IL-1R, MHCII, CD68, CD80, and CD86.
   18. The method of any of items 15-17, wherein a marker characteristic of the pro-inflammatory state comprises one or more of TLR2, TNFα, IL1α, ITAM1, iNOS, IL1β, HIF1α, IL-12b, and KCna3.
   19. The method of any of items 15-18, wherein a marker characteristic of the pro-inflammatory state comprises one or more of GFAP, CLEC7a, and Vimentin.
   20. The method of any of items 15-19, wherein a marker characteristic of the pro-inflammatory state comprises one or more of CD69, CD27, CD45, CD44, and CCR7.
   21. The method of any of items 15-20, wherein a marker characteristic of the anti-inflammatory state or homeostatic state comprises one or more of CD163, MHCII, SR, CD206, CD200R, TGM2, DecoyR, IL-1R, Ym1/2, Fizz1, Arg1, CD86, TLR1, TLR8, VEGF, Arg1, APOE, TIMP2, IGF1, DPP6, P2Rγ12, TMEM119, BIN1, PTGS1, and CD62.
   22. The method of any of items 15-21, wherein a marker characteristic of the anti-inflammatory state or homeostatic state comprises one or more of CD163, MHCII, SR, CD206, CD200R, TGM2, DecoyR, IL-1R, Ym1/2, Fizz1, Arg1, CD86, TLR1, TLR8, and VEGF.
   23. The method of any of items 15-22, wherein a marker characteristic of the anti-inflammatory state or homeostatic state comprises one or more of Arg1, APOE, TIMP2, IGF1, DPP6, P2Rγ12, TMEM119, BIN′, PTGS1, and CD62.
   24. The method of any of items 1-23, wherein the second stimulus is provided to achieve or maintain the anti-inflammatory state of the immune cell.
   25. The method of any of items 1-24, wherein the second stimulus comprises at least one immune-modulating molecule.
   26. The method of any of items 1-25, wherein the at least one immune-modulating molecule comprises an antigen, a cytokine, a growth factor, a sphingolipid, a complement factor, an immunomodulatory small molecule, an intracellular signaling inhibitor, an activator of pro-inflammatory or anti-inflammatory pathways, a cytokine inhibitor, and combinations thereof.
   27. The method of any of items 1-26, wherein a first immune-modulating molecule is administered at the same time as a second immune-modulating molecule.
   28. The method of any of items 1-26, wherein a first immune-modulating molecule is administered before a second immune-modulating molecule.
   29. The method of any of items 1-28, wherein the first immune-modulating molecule is administered between five minutes and 24 hours before the second immune-modulating molecule.
   30. The method of any of items 1-25, wherein a first immune-modulating molecule is different from a second immune-modulating molecule.
   31. The method of any of items 1-25, wherein a first immune-modulating molecule is the same as a second immune-modulating molecule.
   32. The method of any of items 1-31, wherein the system is an open-loop system.
   33. The method of item 32, wherein the detector is configured to detect colorimetric or fluorescent output indicative of the change in the inflammatory state of the immune cell.
   34. The method of items 32 or 33, wherein the change in inflammatory state is measured by a Western blot, ELISA, RNA sequencing, qPCR, qRTPCR, or mass spectrometry.
   35. The method of any of items 1-31, wherein the system is a closed-loop system.
   36. The method of item 35, wherein the detector is configured to detect the change in the inflammatory state of the immune cell in real time.
   37. The method of item 36, wherein the detector is configured to detect colorimetric or fluorescent output indicative of the change in the inflammatory state of the immune cell, and wherein the controller is configured to increase or decrease the amount of the first stimulus or second stimulus in response to the input/output data obtained from the detector.
   38. The method of item 37, wherein the colorimetric or fluorescent output comprises colorimetric or fluorescent reporters of immune marker expression or level.
   39. The method of item 38, wherein the immune marker comprises a cell surface marker or a secreted factor.
   40. The method of any of items 1-39, wherein the fluid chamber further comprises a fluid medium suitable for growth and/or expansion of the immune cell.
   41. A system for dynamic real-time modeling and/or control of an inflammatory response in an immune cell, comprising:

a fluid chamber comprising at least one inlet, at least one outlet, and the immune cell;

a controller in fluid communication with the fluid chamber configured to deliver a first stimulus through the inlet, wherein the stimulus elicits a change in the inflammatory state of the immune cell; and

a detector in fluid communication with the fluid chamber configured to detect the change in the inflammatory state of the immune cell,

wherein the controller is further configured to deliver a second stimulus based on the change in the inflammatory state of the immune cell in order to model and/or control the inflammatory response of the immune cell,

wherein the detector is configured to generate input and/or output data indicative of the change in the inflammatory state of the immune cell, and

wherein the change in the inflammatory state of the immune cell to each of the first stimulus and second stimulus is predicted by the steps of:
fitting a black box engineering model to the input and/or output data obtained by stimulating cells within the chamber; and
selecting a best fitting black box engineering model based on the input and/or output data and applying the best fitting black box engineering model to future input and/or output data.
   42. The system of item 41, wherein the fluid chamber is a cell culture chamber, a cell culture well, or a microfluidic chamber.
   43. The system of items 41 or 42, wherein the immune cell comprises at least one cell selected from the following types of cells: a microglial cell, an astrocyte, a macrophage, a B cell, a T cell, a natural killer (NK) cell, and a leukocyte.
   44. The system of any of items 41-43, wherein the immune cell comprises a microglial cell, a macrophage, or combinations thereof.
   45. The system of any of items 41-44, wherein the first stimulus comprises at least one immune-modulating molecule.
   46. The system of item 45, wherein the at least one immune-modulating molecule is pro-inflammatory or anti-inflammatory.
   47. The system of items 45 or 46, wherein the at least one immune-modulating molecule comprises an antigen, a cytokine, a growth factor, a sphingolipid, a complement factor, an immunomodulatory small molecule, an intracellular signaling inhibitor, an activator of pro-inflammatory or anti-inflammatory pathways, a cytokine inhibitor, and combinations thereof.
   48. The system of any of items 45-47, wherein a first immune-modulating molecule is administered at the same time as a second immune-modulating molecule.
   49. The system of any of items 45-48, wherein a first immune-modulating molecule is administered before a second immune-modulating molecule.
   50. The system of any of items 45-49, wherein the first immune-modulating molecule is administered between five minutes and 24 hours before the second immune-modulating molecule.
   51. The system of any of items 45-50, wherein a first immune-modulating molecule is different from a second immune-modulating molecule.
   52. The system of any of items 45-51, wherein a first immune-modulating molecule is the same as a second immune-modulating molecule.
   53. The system of any of items 41-52, wherein the first stimulus causes the immune cell to change from a pro-inflammatory state to an anti-inflammatory state.
   54. The system of any of items 41-53, wherein the first stimulus causes the immune cell to change from a quiescent state to a pro-inflammatory state.
   55. The system of any of items 41-54, wherein the change in the inflammatory state of the immune cell is detected by measuring a marker characteristic of the inflammatory state.
   56. The system of item 55, wherein a marker characteristic of the pro-inflammatory state comprises one or more of iNOS, SOCS3, TLR4, TLR2, IL-1R, MHCII, CD68, CD80, CD86, TLR2, TNFα, IL1α, ITAM1, IL1β, HIF1α, IL-12b, KCna3, GFAP, CLEC7a, Vimentin, CD69, CD27, CD45, CD44, and CCR7.
   57. The system of items 55 or 56, wherein a marker characteristic of the pro-inflammatory state comprises one or more of iNOS, SOCS3, TLR4, TLR2, IL-1R, MHCII, CD68, CD80, and CD86.
   58. The system of any of items 55-57, wherein a marker characteristic of the pro-inflammatory state comprises one or more of TLR2, TNFα, IL1α, ITAM1, iNOS, IL1β, HIF1α, IL-12b, and KCna3.
   59. The system of any of items 55-58, wherein a marker characteristic of the pro-inflammatory state comprises one or more of GFAP, CLEC7a, and Vimentin.
   60. The system of any of items 55-59, wherein a marker characteristic of the pro-inflammatory state comprises one or more of CD69, CD27, CD45, CD44, and CCR7.
   61. The system of any of items 55-60, wherein a marker characteristic of the anti-inflammatory state or homeostatic state comprises one or more of CD163, MHCII, SR, CD206, CD200R, TGM2, DecoyR, IL-1R, Ym1/2, Fizz1, Arg1, CD86, TLR1, TLR8, VEGF, Arg1, APOE, TIMP2, IGF1, DPP6, P2Rγ12, TMEM119, BIN1, PTGS1, and CD62.
   62. The system of any of items 55-61, wherein a marker characteristic of the anti-inflammatory state or homeostatic state comprises one or more of CD163, MHCII, SR, CD206, CD200R, TGM2, DecoyR, IL-1R, Ym1/2, Fizz1, Arg1, CD86, TLR1, TLR8, and VEGF.
   63. The system of any of items 55-62, wherein a marker characteristic of the anti-inflammatory state or homeostatic state comprises one or more of Arg1, APOE, TIMP2, IGF1, DPP6, P2Rγ12, TMEM119, BIN1, PTGS1, and CD62.
   64. The system of any of items 41-63, wherein the second stimulus is provided to achieve or maintain the anti-inflammatory state of the immune cell.
   65. The system of any of items 41-64, wherein the second stimulus comprises at least one immune-modulating molecule.
   66. The system of any of items 41-65, wherein the at least one immune-modulating molecule comprises an antigen, a cytokine, a growth factor, a sphingolipid, a complement factor, an immunomodulatory small molecule, an intracellular signaling inhibitor, an activator of pro-inflammatory or anti-inflammatory pathways, a cytokine inhibitor, and combinations thereof.
   67. The system of any of items 41-66, wherein a first immune-modulating molecule is administered at the same time as a second immune-modulating molecule.
   68. The system of any of items 41-66, wherein a first immune-modulating molecule is administered before a second immune-modulating molecule.
   69. The system of any of items 41-68, wherein the first immune-modulating molecule is administered between five minutes and 24 hours before the second immune-modulating molecule.
   70. The system of any of items 41-69, wherein a first immune-modulating molecule is different from a second immune-modulating molecule.
   71. The system of any of items 41-69, wherein a first immune-modulating molecule is the same as a second immune-modulating molecule.
   72. The system of any of items 41-71, wherein the system is an open-loop system.
   73. The system of item 72, wherein the detector is configured to detect colorimetric or fluorescent output indicative of the change in the inflammatory state of the immune cell.
   74. The system of items 72 or 73, wherein the change in inflammatory state is measured by a Western blot, ELISA, RNA sequencing, qPCR, qRTPCR, or mass spectrometry.
   75. The system of any of items 41-71, wherein the system is a closed-loop system.
   76. The system of item 75, wherein the detector is configured to detect the change in the inflammatory state of the immune cell in real time.
   77. The system of items 75 or 76, wherein the detector is configured to detect colorimetric or fluorescent output indicative of the change in the inflammatory state of the immune cell, and wherein the controller is configured to increase or decrease the amount of the first stimulus or second stimulus in response to the input/output data obtained from the detector.
   78. The system of any of items 75-77, wherein the colorimetric or fluorescent output comprises colorimetric or fluorescent reporters of immune marker expression or level.
   79. The system of any of items 75-78, wherein the immune marker comprises a cell surface marker or a secreted factor.
   80. The system of any of items 41-79, wherein the fluid chamber further comprises a fluid medium suitable for growth and/or expansion of the immune cell.
   81. A method of treating a disease or condition in a subject in need thereof caused by an aberrant inflammatory response comprising:

monitoring and/or controlling in real time the aberrant inflammatory response in an immune cell, comprising:

providing a fluid chamber comprising at least one inlet, at least one outlet, and the immune cell;

delivering a first stimulus through the inlet via a controller, the controller in fluid communication with the fluid chamber, wherein the stimulus elicits a change in the inflammatory state of the immune cell; and

detecting the change in the inflammatory state of the immune cell via a detector, the detector in fluid communication with the fluid chamber,

wherein the controller is configured to deliver a second stimulus based on the change in the inflammatory state of the immune cell in order to model and/or control the inflammatory response of the immune cell,

wherein the detector is configured to generate input and/or output data indicative of the change in the inflammatory state of the immune cell,

wherein the change in the inflammatory state of the immune cell to each of the first stimulus and second stimulus is predicted by the steps of:
fitting a black box engineering model to the input and/or output data obtained by stimulating cells within the chamber; and
selecting a best fitting black box engineering model based on the input and/or output data and applying the best fitting black box model to future input and/or output data, and

wherein the first and/or second stimulus is administered to the subject in order to control the aberrant inflammatory response thereby treating the disease or condition.

82. The method of item 81, wherein the disease or condition caused by the aberrant immune response comprises an inflammatory disease, such as Alzheimer's disease, Parkinson's disease, frontotemporal dementia, schizophrenia, traumatic brain injury, rheumatoid arthritis, inflammatory bowel disease, chronic obstructive pulmonary disease, and diabetic ulcers.
83. The method of items 81 or 82, wherein the immune cell is obtained from the subject.
84. The method of any of items 81-83, wherein the fluid chamber is a cell culture chamber, a cell culture well, or a microfluidic chamber.
85. The method of any of items 81-84, wherein the immune cell comprises at least one cell selected from the following types of cells: a microglial cell, an astrocyte, a macrophage, a B cell, a T cell, a natural killer (NK) cell, and a leukocyte.
86. The method of any of items 81-85, wherein the immune cell comprises a microglial cell, a macrophage, or combinations thereof.
87. The method of any of items 81-86, wherein the first stimulus comprises at least one immune-modulating molecule.
88. The method of item 87, wherein the at least one immune-modulating molecule is pro-inflammatory or anti-inflammatory.
89. The method of item 87 or 88, wherein the at least one immune-modulating molecule comprises an antigen, a cytokine, a growth factor, a sphingolipid, a complement factor, an immunomodulatory small molecule, an intracellular signaling inhibitor, an activator of pro-inflammatory or anti-inflammatory pathways, a cytokine inhibitor, and combinations thereof.
90. The method of any of items 87-89, wherein a first immune-modulating molecule is administered at the same time as a second immune-modulating molecule.
91. The method of any of items 87-90, wherein a first immune-modulating molecule is administered before a second immune-modulating molecule.
92. The method of any of items 87-91, wherein the first immune-modulating molecule is administered between five minutes and 24 hours before the second immune-modulating molecule.
93. The method of any of items 87-92, wherein a first immune-modulating molecule is different from a second immune-modulating molecule.
94. The method of any of items 87-92, wherein a first immune-modulating molecule is the same as a second immune-modulating molecule.
95. The method of any of items 81-94, wherein the first stimulus causes the immune cell to change from a pro-inflammatory state to an anti-inflammatory state.
96. The method of any of items 81-94, wherein the first stimulus causes the immune cell to change from a quiescent state to a pro-inflammatory state.
97. The method of any of items 81-94, wherein the change in the inflammatory state of the immune cell is detected by measuring a marker characteristic of the inflammatory state.
98. The method of item 97, wherein a marker characteristic of the pro-inflammatory state comprises one or more of iNOS, SOCS3, TLR4, TLR2, IL-1R, MHCII, CD68, CD80, CD86, TLR2, TNFα, IL1α, ITAM1, IL1β, HIF1α, IL-12b, KCna3, GFAP, CLEC7a, Vimentin, CD69, CD27, CD45, CD44, and CCR7.
99. The method of items 97 or 98, wherein a marker characteristic of the pro-inflammatory state comprises one or more of iNOS, SOCS3, TLR4, TLR2, IL-1R, MHCII, CD68, CD80, and CD86.
100. The method of any of items 97-99, wherein a marker characteristic of the pro-inflammatory state comprises one or more of TLR2, TNFα, IL1α, ITAM1, iNOS, IL1β, HIF1α, IL-12b, and KCna3.
101. The method of any of items 97-100, wherein a marker characteristic of the pro-inflammatory state comprises one or more of GFAP, CLEC7a, and Vimentin.
102. The method of any of items 97-101, wherein a marker characteristic of the pro-inflammatory state comprises one or more of CD69, CD27, CD45, CD44, and CCR7.
103. The method of any of items 97-102, wherein a marker characteristic of the anti-inflammatory state or homeostatic state comprises one or more of CD163, MHCII, SR, CD206, CD200R, TGM2, DecoyR, IL-1R, Ym1/2, Fizz1, Arg1, CD86, TLR1, TLR8, VEGF, Arg1, APOE, TIMP2, IGF1, DPP6, P2Rγ12, TMEM119, BIN1, PTGS1, and CD62.
104. The method of any of items 97-103, wherein a marker characteristic of the anti-inflammatory state or homeostatic state comprises one or more of CD163, MHCII, SR, CD206, CD200R, TGM2, DecoyR, IL-1R, Ym1/2, Fizz1, Arg1, CD86, TLR1, TLR8, and VEGF.
105. The method of any of items 97-104, wherein a marker characteristic of the anti-inflammatory state or homeostatic state comprises one or more of Arg1, APOE, TIMP2, IGF1, DPP6, P2Rγ12, TMEM119, BIN1, PTGS1, and CD62.
106. The method of any of items 81-105, wherein the second stimulus is provided to achieve or maintain the anti-inflammatory state of the immune cell.
107. The method of any of items 81-106, wherein the second stimulus comprises at least one immune-modulating molecule.
108. The method of item 107, wherein the at least one immune-modulating molecule comprises an antigen, a cytokine, a growth factor, a sphingolipid, a complement factor, an immunomodulatory small molecule, an intracellular signaling inhibitor, an activator of pro-inflammatory or anti-inflammatory pathways, a cytokine inhibitor, and combinations thereof.
109. The method of items 107 or 108, wherein a first immune-modulating molecule is administered at the same time as a second immune-modulating molecule.
110. The method of any of items 107-109, wherein a first immune-modulating molecule is administered before a second immune-modulating molecule.
111. The method of any of items 107-110, wherein the first immune-modulating molecule is administered between five minutes and 24 hours before the second immune-modulating molecule.
112. The method of any of items 107-111, wherein a first immune-modulating molecule is different from a second immune-modulating molecule.
113. The method of claim any of items 107-111, wherein a first immune-modulating molecule is the same as a second immune-modulating molecule.
114. The method of any of items 81-113, wherein the system is an open-loop system.
115. The method of item 114, wherein the detector is configured to detect colorimetric or fluorescent output indicative of the change in the inflammatory state of the immune cell.
116. The method of items 114 or 115, wherein the change in inflammatory state is measured by a Western blot, ELISA, RNA sequencing, qPCR, qRTPCR, or mass spectrometry.
117. The method of any of items 81-113, wherein the system is a closed-loop system.
118. The method of item 117, wherein the detector is configured to detect the change in the inflammatory state of the immune cell in real time.
119. The method of item 117 or 118, wherein the detector is configured to detect colorimetric or fluorescent output indicative of the change in the inflammatory state of the immune cell, and wherein the controller is configured to increase or decrease the amount of the first stimulus or second stimulus in response to the input/output data obtained from the detector.
120. The method of any of items 117-119, wherein the colorimetric or fluorescent output comprises colorimetric or fluorescent reporters of immune marker expression or level.
121. The method of any of items 117-120, wherein the immune marker comprises a cell surface marker or a secreted factor.
122. The method of any of items 81-121, wherein the fluid chamber further comprises a fluid medium suitable for growth and/or expansion of the immune cell.
123. A method of treating a disease or condition in a subject in need thereof caused by an aberrant inflammatory response comprising:

administering a first stimulus to the subject, wherein the stimulus elicits a change in an inflammatory state of the subject's immune cells;

obtaining a biological sample from the subject;

detecting the change in the inflammatory state via a detector;

delivering a second stimulus based on the change in the inflammatory state of the immune cell in order to model and/or control the inflammatory response of the immune cells,

wherein the detector is configured to generate input and/or output data indicative of the change in the inflammatory state of the immune cells,

wherein the change in the inflammatory state of the immune cells to each of the first stimulus and second stimulus is predicted by the steps of:
fitting a black box engineering model to the input and/or output data obtained by stimulating the subject's immune cells; and
selecting a best fitting black box engineering model based on the input and/or output data and applying the best fitting black box engineering model to future input and/or output data, and

wherein the first and/or second stimulus is administered to the subject in order to control the aberrant inflammatory response thereby treating the disease or condition.

124. The method of item 123, wherein the disease or condition caused by the aberrant immune response comprises an inflammatory disease, such as Alzheimer's disease, Parkinson's disease, frontotemporal dementia, schizophrenia, traumatic brain injury, rheumatoid arthritis, inflammatory bowel disease, chronic obstructive pulmonary disease, and diabetic ulcers.
125. The method of items 123 or 124, wherein the biological sample comprises a biological fluid or tissue.
126. The method of item 125, wherein the biological fluid is selected from the group consisting of blood, serum, plasma, urine, saliva, tears, mucus, lymph, interstitial fluid, cerebrospinal fluid, pus, breast milk, and amniotic fluid.
127. The method of any of items 123-126, wherein the immune cell comprises at least one cell selected from the following types of cells: a microglial cell, an astrocyte, a macrophage, a B cell, a T cell, a natural killer (NK) cell, and a leukocyte.
128. The method of any of items 123-127, wherein the immune cell comprises a microglial cell, a macrophage, or combinations thereof.
129. The method of any of items 123-128, wherein the first stimulus comprises at least one immune-modulating molecule.
130. The method of item 129, wherein the at least one immune-modulating molecule is pro-inflammatory or anti-inflammatory.
131. The method of items 129 or 130, wherein the at least one immune-modulating molecule comprises an antigen, a cytokine, a growth factor, a sphingolipid, a complement factor, an immunomodulatory small molecule, an intracellular signaling inhibitor, an activator of pro-inflammatory or anti-inflammatory pathways, a cytokine inhibitor, and combinations thereof.
132. The method of any of items 129-131, wherein a first immune-modulating molecule is administered at the same time as a second immune-modulating molecule.
133. The method of any of items 129-132, wherein a first immune-modulating molecule is administered before a second immune-modulating molecule.
134. The method of any of items 129-133, wherein the first immune-modulating molecule is administered between five minutes and 24 hours before the second immune-modulating molecule.
135. The method of any of items 129-134, wherein a first immune-modulating molecule is different from a second immune-modulating molecule.
136. The method of any of items 129-135, wherein a first immune-modulating molecule is the same as a second immune-modulating molecule.
137. The method of any of items 123-136, wherein the first stimulus causes the immune cell to change from a pro-inflammatory state to an anti-inflammatory state.
138. The method of any of items 123-136, wherein the first stimulus causes the immune cell to change from a quiescent state to a pro-inflammatory state.
139. The method of any of items 123-138, wherein the change in the inflammatory state of the immune cell is detected by measuring a marker characteristic of the inflammatory state.
140. The method of item 139, wherein a marker characteristic of the pro-inflammatory state comprises one or more of iNOS, SOCS3, TLR4, TLR2, IL-1R, MHCII, CD68, CD80, CD86, TLR2, TNFα, IL1α, ITAM1, IL1β, HIF1α, IL-12b, KCna3, GFAP, CLEC7a, Vimentin, CD69, CD27, CD45, CD44, and CCR7.
141. The method of items 139 or 140, wherein a marker characteristic of the pro-inflammatory state comprises one or more of iNOS, SOCS3, TLR4, TLR2, IL-1R, MHCII, CD68, CD80, and CD86.
142. The method of any of items 139-141, wherein a marker characteristic of the pro-inflammatory state comprises one or more of TLR2, TNFα, IL1α, ITAM1, iNOS, IL1β, HIF1α, IL-12b, and KCna3.
143. The method of any of items 139-142, wherein a marker characteristic of the pro-inflammatory state comprises one or more of GFAP, CLEC7a, and Vimentin.
144. The method of any of items 139-143, wherein a marker characteristic of the pro-inflammatory state comprises one or more of CD69, CD27, CD45, CD44, and CCR7.
145. The method of any of items 139-144, wherein a marker characteristic of the anti-inflammatory state or homeostatic state comprises one or more of CD163, MHCII, SR, CD206, CD200R, TGM2, DecoyR, IL-1R, Ym1/2, Fizz1, Arg1, CD86, TLR1, TLR8, VEGF, Arg1, APOE, TIMP2, IGF1, DPP6, P2Rγ12, TMEM119, BIN1, PTGS1, and CD62.
146. The method of any of items 139-145, wherein a marker characteristic of the anti-inflammatory state or homeostatic state comprises one or more of CD163, MHCII, SR, CD206, CD200R, TGM2, DecoyR, IL-1R, Ym1/2, Fizz1, Arg1, CD86, TLR1, TLR8, and VEGF.
147. The method of any of items 139-146, wherein a marker characteristic of the anti-inflammatory state or homeostatic state comprises one or more of Arg1, APOE, TIMP2, IGF1, DPP6, P2Rγ12, TMEM119, BIN1, PTGS1, and CD62.
148. The method of any of items 123-147, wherein the second stimulus is provided to achieve or maintain the anti-inflammatory state of the immune cell.
149. The method of any of items 123-148, wherein the second stimulus comprises at least one immune-modulating molecule.
150. The method of item 149, wherein the at least one immune-modulating molecule comprises an antigen, a cytokine, a growth factor, a sphingolipid, a complement factor, an immunomodulatory small molecule, an intracellular signaling inhibitor, an activator of pro-inflammatory or anti-inflammatory pathways, a cytokine inhibitor, and combinations thereof.
151. The method of items 149 or 150, wherein a first immune-modulating molecule is administered at the same time as a second immune-modulating molecule.
152. The method of any of items 149-151, wherein a first immune-modulating molecule is administered before a second immune-modulating molecule.
153. The method of any of items 149-152, wherein the first immune-modulating molecule is administered between five minutes and 24 hours before the second immune-modulating molecule.
154. The method of any of items 149-153, wherein a first immune-modulating molecule is different from a second immune-modulating molecule.
155. The method of any of items 149-154, wherein a first immune-modulating molecule is the same as a second immune-modulating molecule.
156. The method of any of items 123-155, wherein the detector is configured to detect immune marker expression or level.
157. The method of item 156, wherein the immune marker comprises a cell surface marker or a secreted factor.
158. The method of items 156 or 157, wherein the immune marker is labeled with a detectable marker comprising a fluorescent marker, a bioluminescent marker, a colorimetric marker, and a radioactive marker.

While several possible embodiments are disclosed above, embodiments of the present disclosure are not so limited. These exemplary embodiments are not intended to be exhaustive or to unnecessarily limit the scope of the disclosure, but instead were chosen and described in order to explain the principles of the present disclosure so that others skilled in the art may practice the disclosure. Indeed, various modifications of the disclosure in addition to those described herein will become apparent to those skilled in the art from the foregoing description. Such modifications are intended to fall within the scope of the appended claims. The scope of the disclosure is therefore indicated by the following claims, rather than the foregoing description and above-discussed embodiments, and all changes that come within the meaning and range of equivalents thereof are intended to be embraced therein.

1. A method comprising:
delivering a first stimulus that elicits a change in the inflammatory state of an immune cell; and
detecting the change in the inflammatory state of the immune cell;
wherein a second stimulus is based on the change in the inflammatory state of the immune cell in order to model and/or control the inflammatory response of the immune cell; and
wherein the change in the inflammatory state of the immune cell to the first stimulus and, if delivered, the second stimulus, is predicted by:
fitting an engineering model to input/output data obtained by stimulating cells; and
selecting the best fitting engineering model based on the input/output data and applying that model to future input/output data.

2. The method of claim 1 further comprising providing a fluid chamber comprising an inlet, an outlet, and the immune cell;
wherein delivering the first stimulus comprises delivering the first stimulus through the inlet via a controller in fluid communication with the fluid chamber;
wherein detecting the change in the inflammatory state comprises detecting the change in the inflammatory state of the immune cell via a detector in fluid communication with the fluid chamber;
wherein the controller is configured to deliver a second stimulus;
wherein the detector is configured to generate the input/output data indicative of the change in the inflammatory state of the immune cell;
wherein fitting the engineering model to the input/output data is obtained by stimulating cells within the fluid chamber; and
wherein the fluid chamber is selected from the group consisting of a cell culture chamber, a cell culture well, and a microfluidic chamber.

3. The method of claim 1, wherein the immune cell comprises one or more of a microglial cell, an astrocyte, a macrophage, a B cell, a T cell, a natural killer (NK) cell, and a leukocyte.

4. The method of claim 1, wherein the method is a method for dynamic real-time modeling and/or control of an inflammatory response in an immune cell; and
wherein the engineering model is a black box engineering model

5. The method of claim 1, wherein the method is a method of treating a disease or condition in a subject in need thereof caused by an aberrant inflammatory response;
wherein the method further comprises:
monitoring and/or controlling in real time the aberrant inflammatory response in the immune cell; and
administering the first and/or second stimulus to the subject in order to control the aberrant inflammatory response thereby treating the disease or condition.

6. The method of claim 2, wherein the first stimulus comprises at least one immune-modulating molecule.

7. The method of claim 6, wherein the at least one immune-modulating molecule comprises an antigen, a cytokine, a growth factor, a sphingolipid, a complement factor, an immunomodulatory small molecule, an intracellular signaling inhibitor, an activator of pro-inflammatory or anti-inflammatory pathways, a cytokine inhibitor, and combinations thereof.

8.-12. (canceled)

13. The method of claim 1, wherein the first stimulus causes the immune cell to change from a pro-inflammatory state to an anti-inflammatory state.

14. The method of claim 1, wherein the first stimulus causes the immune cell to change from a quiescent state to a pro-inflammatory state.

15. The method of claim 1, wherein the change in the inflammatory state of the immune cell is detected by measuring a marker characteristic of the inflammatory state; and
wherein a marker characteristic of a pro-inflammatory state comprises one or more of iNOS, SOCS3, TLR4, TLR2, IL-1R, MHCII, CD68, CD80, CD86, TLR2, TNFα, IL1α, ITAM1, IL113, HIF1α, IL-12b, KCna3, GFAP, CLEC7a, Vimentin, CD69, CD27, CD45, CD44, and CCR7.

16.-20. (canceled)

21. The method of claim 1, wherein the change in the inflammatory state of the immune cell is detected by measuring a marker characteristic of the inflammatory state; and
wherein a marker characteristic of an anti-inflammatory state or homeostatic state comprises one or more of CD163, MHCII, SR, CD206, CD200R, TGM2, DecoyR, IL-1R, Ym1/2, Fizz1, Arg1, CD86, TLR1, TLR8, VEGF, Arg1, APOE, TIMP2, IGF1, DPP6, P2Rγ12, TMEM119, BIN1, PTGS1, and CD62.

22.-34. (canceled)

35. The method of claim 2, wherein the system is a closed-loop system; and
wherein the detector is further configured to:
detect the change in the inflammatory state of the immune cell in real time; and
detect colorimetric or fluorescent output indicative of the change in the inflammatory state of the immune cell, and wherein the controller is configured to increase or decrease the amount of the first stimulus or second stimulus in response to the input/output data obtained from the detector;
wherein the colorimetric or fluorescent output comprises colorimetric or fluorescent reporters of immune marker expression or level.

36.-38. (canceled)

39. The method of claim 35, wherein the immune marker comprises a cell surface marker or a secreted factor.

40. The method of claim 2, wherein the fluid chamber further comprises a fluid medium suitable for growth and/or expansion of the immune cell.

41. A system comprising:
a fluid chamber comprising at least a first inlet, at least a first outlet, and an immune cell;
a controller in fluid communication with the fluid chamber configured to:
deliver a first stimulus through the first inlet, wherein the stimulus elicits a change in the inflammatory state of the immune cell; and
deliver a second stimulus;
wherein the first stimulus causes the immune cell to change from a quiescent state to a pro-inflammatory state, and/or the pro-inflammatory state to an anti-inflammatory state; and
a detector in fluid communication with the fluid chamber configured to:
detect the change in the inflammatory state of the immune cell; and
generate input/output data indicative of the change in the inflammatory state of the immune cell;
wherein the change in the inflammatory state of the immune cell is detected by measuring a marker characteristic of the inflammatory state;
wherein a marker characteristic of the pro-inflammatory state comprises one or more of iNOS, SOCS3, TLR4, TLR2, IL-1R, MHCII, CD68, CD80, CD86, TLR2, TNFα, IL1α, ITAM1, IL113, HIF1α, IL-12b, KCna3, GFAP, CLEC7a, Vimentin, CD69, CD27, CD45, CD44, and CCR7; and
wherein a marker characteristic of the anti-inflammatory state or homeostatic state comprises one or more of CD163, MHCII, SR, CD206, CD200R, TGM2, DecoyR, IL-1R, Ym1/2, Fizz1, Arg1, CD86, TLR1, TLR8, VEGF, Arg1, APOE, TIMP2, IGF1, DPP6, P2Rγ12, TMEM119, BIN1, PTGS1, and CD62;
wherein the second stimulus is based on the change in the inflammatory state of the immune cell in order to model and/or control the inflammatory response of the immune cell; and
wherein the change in the inflammatory state of the immune cell to each of the first stimulus and second stimulus is predicted by:
fitting an engineering model to the input/output data obtained by stimulating cells within the chamber; and
selecting the best fitting engineering model based on the input/output data and applying that model to future input/output data.

42.-74. (canceled)

75. The system of claim 41, wherein the system is a closed-loop system; and
wherein the detector is further configured to:
detect the change in the inflammatory state of the immune cell in real time; and
detect colorimetric or fluorescent output indicative of the change in the inflammatory state of the immune cell, and wherein the controller is configured to increase or decrease the amount of the first stimulus or second stimulus in response to the input/output data obtained from the detector;
wherein the colorimetric or fluorescent output comprises colorimetric or fluorescent reporters of immune marker expression or level.

76.-78. (canceled)

79. The system of claim 75, wherein the immune marker comprises a cell surface marker or a secreted factor.

80. The system of claim 75, wherein the fluid chamber further comprises a fluid medium suitable for growth and/or expansion of the immune cell.

81.-136. (canceled)

137. The method of claim 159, wherein the first modulating stimulus causes the immune cell to change from a pro-inflammatory state to an anti-inflammatory state.

138. The method of claim 159, wherein the first modulating stimulus causes the immune cell to change from a quiescent state to a pro-inflammatory state.

139. The method of claim 159 further comprising detecting the modulation in the inflammatory state of the immune cell by measuring a marker characteristic of the inflammatory state;
wherein a marker characteristic of a pro-inflammatory state comprises one or more of iNOS, SOCS3, TLR4, TLR2, IL-1R, MHCII, CD68, CD80, CD86, TLR2, TNFα, IL1α, ITAM1, IL113, HIF1α, IL-12b, KCna3, GFAP, CLEC7a, Vimentin, CD69, CD27, CD45, CD44, and CCR7; and
wherein a marker characteristic of an anti-inflammatory state or homeostatic state comprises one or more of CD163, MHCII, SR, CD206, CD200R, TGM2, DecoyR, IL-1R, Ym1/2, Fizz1, Arg1, CD86, TLR1, TLR8, VEGF, Arg1, APOE, TIMP2, IGF1, DPP6, P2Rγ12, TMEM119, BIN1, PTGS1, and CD62.

140.-155. (canceled)

156. The method of claim 159 further comprising detecting the modulation in the inflammatory state of the immune cell by measuring immune marker expression or level.

157. The method of claim 156, wherein the immune marker comprises a cell surface marker or a secreted factor.

158. The method of claim 156, wherein the immune marker is labeled with a detectable marker comprising a fluorescent marker, a bioluminescent marker, a colorimetric marker, and a radioactive marker.

159. A method comprising:
retrieving a desired trajectory of immune cell response; and
modulating the inflammatory state of an immune cell to match within a tolerance the desired trajectory of immune cell response;
wherein modulating comprises subjecting the immune cell to at least a first modulating stimulus.

160. The method of claim 159 further comprising determining the desired trajectory of immune cell response.

161. The method of claim 160, wherein determining the desired trajectory of immune cell response comprises:
quantitatively interrogating temporal dynamics of immune cell response of an immune cell to stimuli; and
stochastically modeling the interrogated temporal dynamics to determine the desired trajectory of immune cell response.

162. The method of claim 159 further comprising determining the desired trajectory of immune cell response;
wherein determining the desired trajectory of immune cell response comprises:
quantitatively interrogating temporal dynamics of immune cell response of an immune cell with at least a first stimuli followed in time by a second stimuli;
stochastically modeling the interrogated temporal dynamics to determine the desired trajectory of immune cell response; and
updating the modeling with data indicative of the immune cell response to the modulating.
